SEQUENCE LISTING (2 sequences)

SEQ ID NO: 1 - protein, 7 aa, Artificial Sequence
  Synthetic
  MOD_RES (1)..(1): X is 8-aminooctanoic acid.
XFGWWGC

SEQ ID NO: 2 - protein, 6 aa, Artificial Sequence
  Synthetic
  MOD_RES (1)..(1): X is 8-aminooctanoic acid
  MOD_RES (4)..(4): X is diaminopropionic acid
XFFXDC

(19) United States
(12) Patent Application Publication
(10) Pub. No.: US 2023/0372384 A1
(43) Pub. Date: Nov. 23, 2023

(54) IMMUNOTHERAPY FOR THE TREATMENT OF CANCER

(71) Applicant: TARGIMMUNE THERAPEUTICS AG, Basel (CH)

(72) Inventors: Esteban POMBO-VILLAR, Binningen (CH); Eric KITAS, Aesch (CH); Maya ZIGLER, Basel (CH); Davor BAJIC, Basel (CH); Babette SCHADE, Küssnacht am Rigi (CH); Derrick BROKA, Luzern (CH)

(21) Appl. No.: 18/247,961

(22) PCT Filed: Oct. 7, 2021
(86) PCT No.: PCT/EP2021/077777

§ 371 Date: Apr. 5, 2023

(30) Foreign Application Priority Data
Oct. 8, 2020 (EP) 20200835.5

(51) Int. Cl. A61K 31/713; C07K 16/28; A61P 35/00

(52) U.S. Cl. CPC A61K 31/713; C07K 16/2818; A61P 35/00

(57) ABSTRACT

The present invention relates to a kit-of-parts comprising and a composition comprising a polyplex comprising a double stranded RNA (dsRNA) and a polymeric conjugate comprising a polyethyleneimine (PEI), one or more polyethylene glycol (PEG) moieties and one or more targeting moieties, and wherein each of said one or more targeting moieties is capable of 5 binding to PSMA; and at least one least one immune checkpoint modulator, wherein said at least one least one immune checkpoint modulator is capable of modulating an immune checkpoint protein. Further the invention relates to this composition or kit-of-parts for use in the treatment of cancer.

The present invention relates to the field of immunotherapeutic cancer treatment. Especially, the present invention relates to a kit-of-parts and a composition comprising a polyplex comprising a double stranded RNA (dsRNA) and a polymeric conjugate comprising a polyethyleneimine (PEI), one or more polyethylene glycol (PEG) moieties and one or more targeting moieties, and wherein each of said one or more targeting moieties is capable of binding to PSMA; and at least one immune checkpoint modulator wherein said at least one immune checkpoint modulator is capable of modulating an immune checkpoint protein. Further the invention relates to this composition or kit-of-parts for use in the treatment of cancer, especially prostate cancer.

### RELATED ART

Antibodies that target tumor-associated antigens have become an important treatment modality for malignancies. Several monoclonal antibodies (mAbs) have already proved to be relatively well-tolerated and effective for the treatment of many different malignant diseases. Although these antibodies are commonly used in the clinic, their efficacy is often modest. mAbs must overcome substantial obstacles to reach antigens presented on target cells to be of therapeutic value (Christiansen et al., Mol Cancer Ther, 2004, 3(11), 1493-1501). Moreover, efficiency of antibodies that target tumor-associated antigens is lowered by insufficient activation of the anti-tumor response of the immune system and by inhibition of the immune reaction induced by the tumor itself.

Checkpoint blockade antibodies targeting cytotoxic T-lymphocyte antigen 4 (CTLA-4) and programed cell-death protein 1 (PD-1) have demonstrated acceptable toxicity, promising clinical responses, durable disease control, and improved survival in some patients with advanced melanoma, non-small cell lung cancer (NSCLC), and other tumor types. Engagement of PD-1 by its ligands, either PD-L1 or PD-L2, induces a negative control signal resulting in the inhibition of T cell proliferation, cytokine production, and cytotoxic activity. (Ma et al., Current status and perspectives in translational biomarker research for PD-1/PD-L1 immune checkpoint blockade therapy, Journal of Hematology & Oncology (2016) 9:47).

Cytokines related to tumor necrosis factor (TNF) provide a communication network essential for coordinating multiple cell types into an effective host defense system against pathogens and malignant cells. The tumor necrosis factor superfamily of ligands (TNFSF) and receptors (TNFRSF) provide key communication signals between various cell types during development. TNF receptors (TNFRs) share a conserved ectodomain defined by a cysteine-rich signature. The TNFRs with a co-stimulatory reputation are encoded by genes residing within an immune-response locus in chromosomal region 1p36 and include GITR (glucocorticoid-induced tumor necrosis factor), OX40, 4-1BB, and CD30 (Ward-Kavanagh, et al., The TNF Receptor Superfamily in Co-stimulating and Co-inhibitory Responses, Immunity 44, May 17, 2016).

A different approach for the treatment of malignancies is a vaccine-based therapy. The molecular definition of tumor-associated antigens introduced the possibility of specific vaccines aiming to target the tumor cells. Recombinant vaccines, which are based on peptides or proteins from defined tumor-associated antigens (TAAs) are usually administered together with an adjuvant or an immune modulator. Although these vaccines were able to induce antigen-specific T cell responses, clinical outcomes have been disappointing (Guo et al., Adv Cancer Res, 2013, 119: 421-475).

A further approach in cancer immunotherapy refers to the combination of vaccines with antibodies that activate antitumor immunity by blocking or inhibiting immune checkpoints. Immune checkpoints refer to a plethora of inhibitory pathways hardwired into the immune system that are important for maintaining self-tolerance and modulating the duration and amplitude of physiological immune responses. Tumors co-opt certain immune-checkpoint pathways as a major mechanism of immune resistance. Because many of the immune checkpoints are initiated by ligand-receptor interactions, they can be readily blocked or inhibited by antibodies or modulated by recombinant forms of ligands or receptors. Cytotoxic T-lymphocyte-associated antigen 4 (CTLA-4) antibodies were the first of this class of immunotherapeutics to achieve US Food and Drug Administration (FDA) approval. However, the development of combinatorial approaches of vaccines with antibodies is still a challenge. Combinatorial strategies have to be intelligently designed and guided by mechanistic considerations and preclinical models (Pardoll, Nat Rev Cancer 2012, 12(4), 252-264).

Several novel combinations for immunotherapy in oncology have been suggested (Morrisey et al. Clin. Transl. Sci 2016, 9, 89-104) including non-antigen specific immunotherapy with naked polyIC and blocking antibodies targeting the programmed cell death-1 (PD-1) pathway which was able of inhibiting tumor in cancer mouse models of B16 melanoma, Lewis lung carcinoma, and MC38 colon carcinoma (Nagato et al., OncoImmunology 2014, 3: e28440).

Despite first encouraging results, the development of cancer immunotherapies continues to be a major challenge for tumor immunologists. Thus, there is a high need for an effective and well-tolerated immunotherapy for the treatment of cancer, especially prostate cancer.

### SUMMARY OF THE INVENTION

The present invention provides a novel combinatorial immunotherapeutic approach for the treatment of cancer.

In a first aspect, the invention relates to a kit-of-parts or composition comprising

a. a polyplex comprising a double stranded RNA (dsRNA) and a polymeric conjugate, wherein said polymeric conjugate comprises a polyethyleneimine (PEI), one or more polyethylene glycol (PEG) moieties and one or more targeting moieties, wherein said PEI is covalently bound to one or more PEG moieties, and each of said one or more PEG moieties is linked to one of said one or more targeting moieties, and wherein each of said one or more targeting moieties is capable of binding to prostate specific membrane antigen (PSMA); and

b. at least one immune checkpoint modulator, wherein said immune checkpoint modulator is capable of modulating an immune checkpoint protein.

In a further aspect, the invention relates to the composition or the kit-of-parts for use in the treatment of cancer. The inventors surprisingly found that combination treatment according to the invention with (i) the polyplex comprising double stranded RNA (dsRNA) and the polymeric conjugate comprising one or more targeting moieties capable of binding to PSMA, and (ii) one or more immune checkpoint modulators capable of modulating an immune checkpoint protein enforces activity of the immune system and leads to potent antitumor activity. Antitumor activity of the immune system was more enhanced by the combination of (i) the PSMA binding polyplex comprising dsRNA and the polymeric conjugate plus (ii) one or more immune checkpoint modulators as compared to the polyplex alone. In some individuals the combination of the polyplex and the at least one immune checkpoint modulator generated a sustained tumor defense against the cancer cells.

Exposing immune cells, preferably peripheral blood mononuclear cells (PBMCs), to medium from PSMA expressing or overexpressing cells that had been treated with the polyplex of the invention, or culturing said immune cells in the presence of cells treated with the polyplex of the invention induced the PBMCs to secrete interferon. Adding an immune checkpoint modulator thereto led to a further significant increase in interferon secretion (cf. FIGS. 3 and 4).

Thus, the combination of the PSMA targeted polyplex and at least one immune checkpoint modulator in accordance with the present invention is capable of broadening the efficacy of the immunomodulatory antibodies to cancer patients, especially prostate cancer patients that are currently not showing any response. Utilizing PSMA targeted delivery of dsRNA, preferably polyIC in combination with anti-checkpoint antibodies shows significant efficacy due to the capabilities of the inventive compositions and kits-of-parts to reinstate the immune system against the prostate tumor.

### DESCRIPTION OF FIGURES

FIG. 1: IP-10 (CXCL10) secretion from cancer cells following treatment with PEI-PEG-DUPA/polyIC. LNCaP and MCF7 cells (40,000 cells per well) were treated for 5 hours with PEI-PEG-DUPA/polyIC at various concentrations (0.125, 0.25, 0.5, 1.0 μg/ml), and human IP-10 secreted to the medium was measured by ELISA. Human IP-10 secretion increased in PSMA overexpressing cells, LNCaP, compared to MCF7 cells which do not express PSMA. IP-10 secretion was induced following treatment with PEI-PEG-DUPA/polyIC in LNCaP cells, which overexpress PSMA, but not in MCF7 cells, which do not express PSMA. LNCaP is a human prostate adenocarcinoma cell line commonly used in the field of oncology.

FIG. 2: Flow cytometry analysis of PD-L1 expression on LNCaP cells following treatment with PEI-PEG-DUPA/polyIC. LNCaP cells were treated for 5 hours with PEI-PEG-DUPA/polyIC at concentrations of 0.031 μg/ml, 0.063 μg/ml, or 0.125 μg/ml and then subjected to flow cytometry, using PE-conjugated anti-PD-L1 antibody or IgG1 isotype control. PD-L1 expression, indicated by Mean Fluorescence Intensity (MFI) increased after PEI-PEG-DUPA/polyIC treatment 0.031p g/ml, 0.063p g/ml, or 0.125 μg/ml in a dose-dependent manner (MFI=695, 725, and 788 respectively) compared to untreated control cells (MFI=579). Isotype control used as negative control (MFI=153).

FIG. 3: IFN-γ secretion from PBMCs co-cultured with LNCaP cancer cells, following combined treatment with anti-4-1BB antibody and PEI-PEG-DUPA/polyIC. PBMCs (unstimulated or stimulated with anti-CD3 antibody (5 ng/ml)) were co-cultured with LNCaP cells in the presence or absence of PEI-PEG-DUPA/polyIC (0.125 μg/ml) and/or an antibody against 4-1BB (10 μg/ml). After overnight incubation, the medium was collected and IFN-γ was measured by ELISA. Combining PEI-PEG-DUPA/polyIC polyplex with anti-4-1BB antibody led to a 2-fold increase in IFN-7 secretion by anti-CD3-stimulated PBMCs, as compared to either single treatment, indicating that the combination treatment led to stronger PBMC activation, as demonstrated by IFN-7 ELISA. PBMCs were not stimulated or stimulated with anti-CD3 antibody (5 ng/ml) and co-cultured with LNCaP cells treated with antibodies against 4-1BB (10 μg/ml) alone or in combination with PEI-PEG-DUPA/polyIC (0.125 μg/ml) ((+)4-1BB). As a control PBMCs were not stimulated or stimulated with anti-CD3 antibody (5 ng/ml) and co-cultured with LNCaP cells untreated or treated with PEI-PEG-DUPA/polyIC (0.125 μg/ml) ((−)4-1BB). After overnight incubation, IFN-γ ELISA was performed to quantify PBMC activation. Fold change of IFN-γ is presented and was compared to untreated PBMCs from each set ((+)4-1BB or (−)4-1BB). Statistics (one-way ANOVA test followed by Tukey's multiple comparisons test) results in a significant difference of PEI-PEG-DUPA/polyIC (+)4-1BB. As a control PBMCs were not stimulated or stimulated with anti-CD3 antibody (5 ng/ml) p Value: between ≤0.0001

FIG. 4: Combination of PEI-PEG-DUPA/polyIC polyplex with Nivolumab, an anti-PD-1 antibody, led to increased PBMC activation, as demonstrated by IFN-γ ELISA. LNCaP cells or medium alone (in the absence of any cells) were untreated or treated with PEI-PEG-DUPA/polyIC polyplex (0.125 μg/ml) for 5 hours. PBMCs were either unstimulated or stimulated with anti-CD3 antibody (500 ng/ml) and treated with or without Nivolumab (20 μg/ml). Then, supernatant from PEI-PEG-DUPA/polyIC-polyplex-treated or untreated (UT) LNCaP cancer cells (“LNCaP Supernatant”) or PEI-PEG-DUPA/polyIC polyplex in medium or untreated medium (“Medium alone”) was transferred to the PBMCs. After overnight incubation, IFN-γ ELISA was performed to quantify PBMC activation. Anti-CD3-stimulated PBMCs treated with both Nivolumab and supernatant from PEI-PEG-DUPA/polyIC-treated LNCaP cells secreted significantly more IFN-γ than those with either single treatment. Thus, the combination treatment led to stronger PBMC activation. Statistics: Multiple t-tests, statistical significance determined using the Holm-Sidak method p Value: **≤0.01; ****≤0.0001

### DETAILED DESCRIPTION OF THE INVENTION

Unless defined otherwise, all technical and scientific terms used herein have the same meanings as commonly understood by one of ordinary skill in the art to which this invention belongs.

Throughout this specification and the claims which follow, unless the context requires otherwise, the word “comprise” or the word “include”, and variations such as “comprises/includes” and “comprising/including”, are to be understood to imply the inclusion of an element, stated integer, step or a group thereof but not the exclusion of any other element, stated integer, step or a group thereof.

As used in this specification and the appended claims, the singular forms “a”, “an”, and “the” include plural referents, unless the content clearly dictates otherwise.

The term “about” or “approximately” when used in connection with a numerical value is meant to encompass numerical values within a range having a lower limit that is 0-10% smaller than the indicated numerical value and having an upper limit that is 0-10% larger than the indicated numerical value. The term “about” or “approximately” means preferably +10%, more preferably +5%, again more preferably +3% or most preferably +0% (referring to the given numeric value, respectively). In each of the invention embodiments, “about” can be deleted. All ranges of values disclosed herein, should refer and include to any and all values falling within said range including the values defining the range.

In one aspect, the invention refers to a composition comprising

a. a polyplex comprising a double stranded RNA (dsRNA) and a polymeric conjugate, wherein said polymeric conjugate comprises a polyethyleneimine (PEI), one or more polyethylene glycol (PEG) moieties and one or more targeting moieties, wherein said PEI is covalently bound to one or more PEG moieties, and each of said one or more PEG moieties is linked to one of said one or more targeting moieties, and wherein each of said one or more targeting moieties is capable of binding to prostate specific membrane antigen (PSMA); and

b. at least one immune checkpoint modulator, wherein said at least one immune checkpoint modulator is capable of modulating an immune checkpoint protein.

In a further aspect, the invention refers to a kit-of-parts comprising

a. a composition, wherein said composition comprises a polyplex comprising a double stranded RNA (dsRNA) and a polymeric conjugate, wherein said polymeric conjugate comprises a polyethyleneimine (PEI), one or more polyethylene glycol (PEG) moieties and one or more targeting moieties, wherein said PEI is covalently bound to one or more PEG moieties, and each of said one or more PEG moieties is linked to one of said one or more targeting moieties, and wherein each of said one or more targeting moieties is capable of binding to prostate specific membrane antigen (PSMA); and

b. at least one immune checkpoint modulator, wherein said immune checkpoint modulator is capable of modulating an immune checkpoint protein.

In a preferred embodiment, said composition of the invention comprises at least one pharmaceutically acceptable diluent, excipient or carrier. In a particularly preferred embodiment, the composition according to the invention is a fixed-dose composition that comprises the polyplex and one or more immunomodulatory antibodies in a single dosage form. In another preferred embodiment, said pharmaceutical composition includes one or more adjuvants.

The term “kit-of-parts” as used herein preferably refers to a combination of at least two separate parts, namely said polyplex and said one or more immunomodulatory antibodies. In a preferred embodiment, said composition is a pharmaceutical composition. The arrangement and construction of such kit-of-parts is conventionally known to one skilled in the art. In a particularly preferred embodiment, said kit-of-parts of the invention or said parts of the kit-of-parts of the invention, i.e. the polyplex and/or the one or more immunomodulatory antibodies comprise independently of each other at least one pharmaceutically acceptable diluent, excipient or carrier. In another preferred embodiment, said kit-of-parts of the invention or said parts of the kit-of-parts of the invention, i.e. the polyplex and/or the one or more immunomodulatory antibodies comprise independently of each other one or more adjuvants.

In certain embodiments, the composition and kit-of-parts according to the invention is formulated for administration by any known method. The composition and kit-of-parts according to the invention, i.e. the polyplex and/or the one or more immunomodulatory antibodies, and the pharmaceutical composition may be formulated for any suitable route of administration including but not limited to intravenous, intra-brain (intracerebral), oral, intramuscular, subcutaneous, transdermal, intradermal, transmucosal, intranasal, sublingual, intraperitoneal or intraocular administration.

In another more preferred embodiment, said composition or kit-of-parts according to the invention is formulated for systemic administration. Again, more preferably, the composition and kit-of-parts according to the invention, i.e. the polyplex and/or the one or more immunomodulatory antibodies, are formulated for intravenous, intraperitoneal, or subcutaneous administration. More preferably, the composition and kit-of-parts according to the invention, i.e. the polyplex and/or the one or more immunomodulatory antibodies, are formulated as one or more dosage forms suitable for injection, preferably as solution, emulsion or suspension suitable for injection.

In one embodiment, the composition and kit-of-parts according to the invention comprising the polyplex of the invention and one or more immunomodulatory antibodies, wherein said polyplex and said one or more immunomodulatory antibodies are present in the composition and kit-of-parts in a therapeutically effective amount.

Said kit-of-parts of the invention may include containers that contain the polyplex and/or the one or more antibodies and/or an apparatus for administering the parts of the kit, i.e. the polyplex and/or the one or more antibodies. In a preferred embodiment, said kit-of-parts of the invention comprises at least one container comprising an effective dose of said polyplex and at least one container comprising an effective dose of said one or more antibodies, and optionally an instruction leaflet.

In a preferred embodiment, said one or more targeting moieties capable of binding to prostate specific membrane antigen (PSMA) are PSMA antibodies, PSMA aptamers or small-molecule PSMA targeting moieties.

In some embodiments, the PSMA targeting moiety is an anti-PSMA antibody, i.e. an antibody that binds to PSMA. Such PSMA antibodies include, but are not limited to, scFv antibodies A5, G0, G1, G2, and G4 and mAbs 3/E7, 3/F11, 3/A12, K7, K12, and D20 (Elsasser-Beile et al., 2006, Prostate, 6611359); mAbs E99, J591, J533, and J415 (Liu et al., 1997, Cancer Res., 5713629; Liu et al., 1998, Cancer Res., 5814055; Fracasso et al., 2002, Prostate, 5319; McDevitt et al., 2000, Cancer Res., 6016095; McDevitt et al., 2001, Science, 29411537; Smith-Jones et al., 2000, Cancer Res., 6015237; Vallabhajosula et al., 2004, Prostate, 581145; Bander et al., 2003, J. Urol., 17011717; Patri et al., 2004, Bioconj. Chem., 1511174; and U.S. Pat. No. 7,163,680); mAb 7E11-C5.3 (Horoszewicz et al., 1987, Anticancer Res., 7:927); antibody 7E11 (Horoszewicz et al., 1987, Anticancer Res., 7 1927; and U.S. Pat. No. 5,162,504); and antibodies described in Chang et al., 1999, Cancer Res., 5913192; Murphy et al., 1998, J. Urol., 16012396; Grauer et al., 1998, Cancer Res., 5814787; and Wang et al., 2001, Int. J. Cancer, 92:871. All of the foregoing documents (scientific and other publications, patents and patent applications) are incorporated herein by reference in their entirety.

More preferably, said one or more targeting moieties capable of binding to PSMA is a small molecule PSMA targeting moiety, again more preferably a small molecule PSMA targeting peptidase inhibitor. In a preferred embodiment, said small molecule PSMA peptidase inhibitors include 2-PMPA, GPI5232, VA-033, phenylalkylphosphonamidates (Jackson et al., 2001, Curr. Med. Chem., 8:949; Bennett et al., 1998, J. Am. Chem. Soc., 120112139; Jackson et al., 2001, J Med. Chem., 4414170; Tsukamoto et al., 2002, Bioorg. Med. Chem. Lett., 1212189; Tang et al., 2003, Biochem. Biophys. Res. Commun., 307:8; Oliver et al., 2003, Bioorg. Med. Chem., 1114455; and Maung et al., 2004, Bioorg. Med. Chem., 1214969), and/or analogs and derivatives thereof. All of the foregoing documents (scientific and other publications, patents and patent applications) are incorporated herein by reference in their entirety.

In some embodiments, said small molecule PSMA targeting moieties are proteins, peptides, amino acids or derivatives thereof. In a preferred embodiment, said small molecule PSMA targeting moiety includes thiol and indole thiol derivatives, such as 2-MPPA and 3-(2-mercaptoethyl)-1H-indole-2-carboxylic acid derivatives (Majer et al., 2003, J Med. Chem., 4611989; and U.S. Patent Publication 2005/0080128). In some embodiments, said small molecule PSMA targeting moieties comprise hydroxamate derivatives (Stoerrner et al., 2003, Bioorg. Med. Chem. Lett., 1312097). In a preferred embodiment, said small molecule PSMA peptidase inhibitors include androgen receptor targeting agents (ARTAs), such as those described in U.S. Pat. Nos. 7,026,500; 7,022,870; 6,998,500; 6,995,284; 6,838,484; 6,569,896; 6,492,554; and in U.S. Patent Publications 2006/0287547; 2006/0276540; 2006/0258628; 2006/0241180; 2006/0183931; 2006/0035966; 2006/0009529; 2006/0004042; 2005/0033074; 2004/0260108; 2004/0260092; 2004/0167103; 2004/0147550; 2004/0147489; 2004/0087810; 2004/0067979; 2004/0052727; 2004/0029913; 2004/0014975; 2003/0232792; 2003/0232013; 2003/0225040; 2003/0162761; 2004/0087810; 2003/0022868; 2002/0173495; 2002/0099096; 2002/0099036. In some embodiments, said small molecule PSMA targeting moieties include polyamines, such as putrescine, spermine, and spermidine (U.S. Patent Publications 2005/0233948 and 2003/0035804). All of the foregoing documents (scientific and other publications, patents and patent applications) are incorporated herein by reference in their entirety.

In a preferred embodiment, said small molecule PSMA peptidase inhibitors include PBDA- and urea-based inhibitors, such as ZJ 43, ZJ 11, ZJ 17, ZJ 38 (Nan et al., 2000, J. Med. Chem., 431772; and Kozikowski et al., 2004, J. Med. Chem., 4711729), and/or and analogs and derivatives thereof. In a preferred embodiment, said one or more targeting moieties capable of binding to prostate specific membrane antigen (PSMA) are small-molecule PSMA targeting moieties, more preferably small urea-based inhibitors.

In preferred embodiments, said molecule PSMA targeting moieties are urea-based inhibitors (herein also called urea-based peptidase inhibitors), more preferably small urea-based inhibitors, such as disclosed in Kularatne et al., Mol Pharmaceutics 2009, 6, 780; Kularatne et al., Mol. Pharmaceutics 2009, 6, 790; Kopka et al., J Nucl Med 2017, 58:17S-26S, Kozikowski et al., J Med Chem. 2001, 44:298-301, Kozikowski et al., J Med Chem. 2004, 47:1729-1738, WO2017/044936, WO2011/084518, WO2011/084521, WO2011/084513, WO2012/166923, WO2008/105773, WO2008/121949, WO2012/135592, WO2010/005740, WO2015/168379, WO03/045436, WO03/045436, WO2016/1 83 447, US 2015/258102, WO2011/084513, WO 2017/089942, US2010/278927, WO2012/016188, WO2008/124634, WO2009/131435, US 2007/225213, WO2017/086467, WO2009/026177, WO2012005572, WO2014/072357, and WO2011/108930. All of the foregoing documents (scientific and other publications, patents and patent applications) are incorporated herein by reference in their entirety.

The term “small molecule” as used herein relates to an organic molecule that has a molecular weight of less than about 2000 g/mol. In some embodiments, the small molecule has a molecular weight of less than about 1500 g/mol, more preferably less than about 1000 g/mol. In a further preferred embodiment, the small molecule has a molecular weight of less than about 800 g/mol, again more preferably less than about 500 g/mol.

In a preferred embodiment, said one or more targeting moieties capable of binding to prostate specific membrane antigen (PSMA) are dipeptide urea based PSMA peptidase inhibitors, more preferably, small molecule dipeptide urea-based PSMA peptidase inhibitors. The term “urea based PSMA peptidase inhibitors” relate to PSMA peptidase inhibitors comprising a urea group. The term “dipeptide urea based PSMA peptidase inhibitors” relate to PSMA peptidase inhibitors comprising a urea group and two peptides or amino acids each independently attached to the —NH₂ groups of the urea group. PSMA peptidase inhibitors reduce activity of the PSMA transmembrane zinc(II) metalloenzyme that catalyzes the cleavage of terminal glutamates. More preferably, said small molecule urea-based PSMA peptidase inhibitor has a molecular weight of less than about 500 g/mol. Again more preferably, said small molecule urea-based PSMA peptidase inhibitor is a Glutamate-urea based PSMA peptidase inhibitor, preferably such as mentioned in Kopka et al., J Nuc Med, 58(9), suppl. 2, 2017; Wirtz et al., EJNMMI Research (2018) 8:84 and references cited therein, all incorporated herein by reference in their entirety.

In a preferred embodiment, said urea based PSMA peptidase inhibitors is a glutamate-urea moiety of formula I, preferably of formula I*:

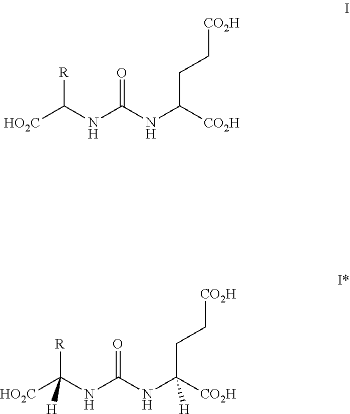

wherein R is C1-6-alkyl, preferably C2-C4-alkyl, substituted one or more times, preferably one time with OH, SH, NH₂, or COOH, wherein one of said NH₂, OH or SH or COOH group serve as the point of covalent attachment for linking to the PEG moiety and wherein the alkyl group is optionally be interrupted by N(H), S or O. In another preferred embodiment, R is C1-6-alkyl, preferably C2-C4-alkyl, substituted one time with OH, SH, NH₂, or COOH, wherein said NH₂, OH, or SH or COOH group serve as the point of covalent attachment for linking to the PEG moiety. In a very preferred embodiment, R is C2-alkyl substituted one time with COOH, wherein said COOH group serve as the point of covalent attachment for linking to the PEG moiety. In a preferred embodiment, said one or more PEG moieties are directly linked to one of said one or more targeting moieties or linked to one of said one or more targeting moieties via one or more linkers.

In a preferred embodiment, said one or more targeting moieties is a glutamate-urea moiety of formula I:

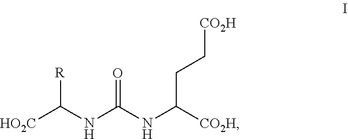

wherein R is C1-6-alkyl, preferably C2-C4-alkyl, substituted one or more times, preferably one time with OH, SH, NH₂, or COOH, wherein one of said NH₂, OH or SH or COOH group serve as the point of covalent attachment for linking to the PEG moiety and wherein the alkyl group is optionally be interrupted by N(H), S or O. In another preferred embodiment, R is C1-6-alkyl, preferably C2-C4-alkyl, substituted one time with OH, SH, NH₂, or COOH, wherein said NH₂, OH, or SH or COOH group serve as the point of covalent attachment for linking to the PEG moiety. In a very preferred embodiment, R is C2-alkyl substituted one time with COOH, wherein said COOH group serve as the point of covalent attachment for linking to the PEG moiety. In a preferred embodiment, said one or more PEG moieties are directly linked to one of said one or more targeting moieties or linked to one of said one or more targeting moieties via one or more linkers.

In another preferred embodiment, said one or more targeting moieties is a glutamate-urea moiety of formula I*

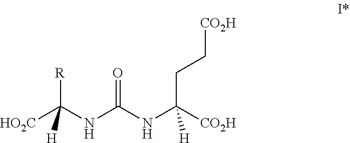

wherein R is C1-6-alkyl, preferably C2-C4-alkyl, substituted one or more times, preferably one time with OH, SH, NH₂, or COOH, wherein one of said NH₂, OH or SH or COOH group serve as the point of covalent attachment for linking to the PEG moiety and wherein the alkyl group is optionally be interrupted by N(H), S or O. In another preferred embodiment, R is C1-6-alkyl, preferably C2-C4-alkyl, substituted one time with OH, SH, NH₂, or COOH, wherein said NH₂, OH, or SH or COOH group serve as the point of covalent attachment for linking to the PEG moiety. In a very preferred embodiment, R is C2-alkyl substituted one time with COOH, wherein said COOH group serve as the point of covalent attachment for linking to the PEG moiety. In a preferred embodiment, said one or more PEG moieties are directly linked to one of said one or more targeting moieties or linked to one of said one or more targeting moieties one or more linkers.

The term “alkyl”, as used herein, refers to a straight or branched hydrocarbon chain radical, preferably a straight or branched hydrocarbon chain radical, consisting solely of carbon and hydrogen atoms, containing no unsaturation, having typically and preferably from one to six carbon atoms (e.g., (C₁₋₆alkyl). Whenever it appears herein, a numerical range such as “1 to 6” refers to each integer in the given range. For example, “1 to 6 carbon atoms” means that the alkyl group may consist of 1 carbon atom, 2 carbon atoms, 3 carbon atoms, etc., up to and including 6 carbon atoms, although the definition is also intended to cover the occurrence of the term “alkyl” where no numerical range is specifically designated. Typical alkyl groups include, but are not limited to methyl, ethyl, n-propyl, prop-2-yl, n-butyl, but-2-yl, 2-methyl-prop-1-yl or 2-methyl-prop-2-yl.

In a further preferred embodiment, said one or more targeting moieties comprises or preferably consists of a DUPA derivative (HOOC(CH₂)₂—CH(COOH)—NH—CO—NH—CH(COOH)—CH₂—) or DUPA moiety (HOOC(CH₂)₂—CH(COOH)—NH—CO—NH—CH(COOH)—(CH₂)₂—CO—).

In a further very preferred embodiment, said one or more targeting moieties comprises or preferably consists of a DUPA moiety (HOOC(CH₂)₂—CH(COOH)—NH—CO—NH—CH(COOH)—(CH₂)₂—CO—). In a preferred embodiment, said one or more PEG moieties are directly linked to one of said one or more targeting moieties or linked to one of said one or more targeting moieties via one or more linkers.

In a further very preferred embodiment, said one or more targeting moieties consists of a DUPA moiety (HOOC(CH₂)₂—CH(COOH)—NH—CO—NH—CH(COOH)—(CH₂)₂—CO—). In a preferred embodiment, said one or more PEG moieties are directly linked to one of said one or more targeting moieties or linked to one of said one or more targeting moieties via one or more linkers.

In a further very preferred embodiment, said one or more targeting moieties consists of a DUPA moiety (HOOC(CH₂)₂—CH(COOH)—NH—CO—NH—CH(COOH)—(CH₂)₂—CO—), wherein both chiral C-atoms having (S)-configuration, as depicted in formula I*. In a preferred embodiment, said one or more PEG moieties are directly linked to one of said one or more targeting moieties or linked to one of said one or more targeting moieties via one or more linkers.

Preferably, said one or more targeting moieties capable of binding to prostate specific membrane antigen (PSMA) or said small molecule urea-based PSMA peptidase inhibitor comprises a DUPA derivative defined herein as HOOC(CH₂)₂—CH(COOH)—NH—CO—NH—CH(COOH)—CH₂—, or a DUPA moiety defined herein as HOOC(CH₂)₂—CH(COOH)—NH—CO—NH—CH(COOH)—(CH₂)₂—CO—. Most preferably, said one or more targeting moieties capable of binding to prostate specific membrane antigen (PSMA) or said small molecule urea-based PSMA peptidase inhibitor consists of the DUPA moiety (HOOC(CH₂)₂—CH(COOH)—NH—CO—NH—CH(COOH)—(CH₂)₂—CO—) or the DUPA derivative (HOOC(CH₂)₂—CH(COOH)—NH—CO—NH—CH(COOH)—CH₂—).

In a preferred embodiment, said DUPA moiety or said DUPA derivative is linked to said one or more PEG moieties either directly or preferably via a linker. Preferably, said linker is as disclosed e.g. in US2020/0188523A1, US2011/0288152A1, US2010/324008A1, all patent applications incorporated herein by reference in its entirety. Preferably, said linker is a peptide linker or a C1-C10 alkylene linker or a combination of both. More preferably, said linker is a peptide linker.

More preferably, said peptide linker is of SEQ ID NO: 1 (—(NH—(CH₂)₇—CO)-Phe-Phe-(NH—CH₂—CH(NH₂)—CO)-Asp-Cys-) or SEQ ID NO: 2 (—(NH—(CH₂)₇—CO)-Phe-Gly-Trp-Trp-Gly-Cys-). Again more preferably, said peptide linker is of SEQ ID NO: 2 (—(NH—(CH₂)₇—CO)-Phe-Gly-Trp-Trp-Gly-Cys-). In a further preferred embodiment, said linker is of SEQ ID NO: 1 or 2 and the targeting moiety is HOOC(CH₂)₂—CH(COOH)—NH—CO—NH—CH(COOH)—(CH₂)₂—CO— (DUPA moiety). More preferably, said linker is of SEQ ID NO: 2 and the targeting moiety is HOOC(CH₂)₂—CH(COOH)—NH—CO—NH—CH(COOH)—(CH₂)₂—CO— (DUPA moiety).

The term “immune checkpoint protein” or “immune checkpoint” is known and described in the art (see for instance Pardoll, 2012, Nature Rev Cancer 12: 252-264; Darvin et al., 2018, Experimental & Molecular Medicine 50:165) and includes protein regulators of the immune system that inhibit or activate (co-stimulate) the immune system, especially T cells.

In a preferred embodiment, said immune checkpoint protein refers to receptors of T cells and dendritic cells, B cells, natural killer (NK) cells, neutrophils and macrophages as well as their soluble or bound ligands and counter-receptors that can stimulate or inhibit activity of the immune system. In a further preferred embodiment, said immune checkpoint protein refers to receptors of T cells and NK cells as well as their soluble or bound ligands and counter-receptors that can co-stimulate or co-inhibit activity of the immune system. Preferably, said activity of the immune system is detected by measuring a T cell response, as shown herein. Preferably, the immune checkpoint protein is a human immune checkpoint protein.

Immune checkpoints refer to inhibitory and activating proteins of the immune system that are crucial for maintaining self-tolerance and modulating the duration and amplitude of physiological immune responses in order to minimize collateral tissue damage. Tumors co-opt certain immune-checkpoint pathways as a major mechanism of immune resistance, particularly against T cells that are specific for tumor antigens. T cells have been the major focus of efforts to therapeutically manipulate endogenous anti-tumor immunity owing to: their capacity for the selective recognition of peptides derived from proteins in all cellular compartments; their capacity to directly recognize and kill antigen-expressing cells (by CD8+ effector T cells; CTLs); and their ability to orchestrate diverse immune responses (by CD4+ helper T cells), which integrates adaptive and innate effector mechanisms. Thus, agonists of co-stimulatory receptors or antagonists of inhibitory signals, both of which result in the amplification of antigen-specific T cell responses, are the agents in current clinical testing. Because many of the immune checkpoints are initiated by ligand-receptor interactions, they can be readily modulated by immune checkpoint modulators.

The kit-of-parts or composition of the invention comprises (a) the polyplex, and (b) at least one immune checkpoint modulator. The term “immune checkpoint modulator” as used herein relates to a compound that is capable of modulating the function of an immune checkpoint protein and promoting activity of the immune system, especially T cells. The term “immune checkpoint modulator” includes (i) agonistic immune checkpoint modulators of co-stimulatory checkpoint proteins and their ligands and (ii) antagonistic immune checkpoint modulators of inhibitory checkpoint proteins and their ligands, all of which result in the promotion of the immune system. Promotion of immune system activity includes generation of enhanced immune responses to an antigen and/or reduction in immunosuppressive immune responses against the antigen. Preferably, promotion of immune system activity results in antigen-specific T cell responses and immune-mediated elimination of tumor cells. The term “immune checkpoint modulator” includes compounds selected from RNA, DNA or peptide aptamers, antibodies, receptor ligands, antibody mimetics, e.g., ankyrin repeats (DARPins), adnectins, affibodies, anti-calins, or engineered Kunitz-type inhibitors etc. In a preferred embodiment, said immune checkpoint modulator is an antibody, a DARPin or a single-stranded oligonucleotide aptamer.

The term “modulating” (or “modulator”) includes activation, which relates to functional stimulation or enhancement of a co-stimulatory immune checkpoint protein as well as inhibition of a co-inhibitory immune checkpoint protein which relates to reduction in activity and full blockade of a co-inhibitory immune checkpoint protein.

The designation “modulating activity of an immune checkpoint protein” or “modulating an immune checkpoint protein” includes stimulation of T cells including T helper cells, CTLs natural killer T cells, natural killer cells (NK), B cells, monocytes, macrophages, and dendritic cells. Stimulation (or activation) induced by modulation of an immune checkpoint protein is preferably detected via measuring increased levels of cytokines such as interferon, especially IFN-γ, produced or released, in particular, by T and NK cells as compared to controls without administration of immune checkpoint protein modulating antibodies, as herein demonstrated via ELISA assays on IFN-γ secretion of PBMCs.

Examples of immune checkpoint proteins include, without limitation, and are preferably selected from the group consisting of PD-1 (Programmed Death 1, interchangeable used herein and equivalent to PD-1, PD-L1, PD-L2, CTLA-4/B7-1/CD152 (Cytotoxic T-Lymphocyte-Associated protein 4), CD137/4-1BB, 4-1BB ligand (4-1BBL), TIM-3 (T-cell Immunoglobulin domain and Mucin domain 3)/HAVCR2, LAG-3, By-He, H4, IDO1, CD40/TNFRSF5, CD40 ligand, OX40/CD134, OX-40 ligand/OX-40L, GITR (Glucocorticoid-Induced TNFR family Related gene)/TNFRSF18, GITR ligand/TNFSF18, ICOS/AILM/CD278, CD122, CD155/PVR, CD226/DNAM-1, CD27, TNFSF14/LIGHT/CD258, CD70/CD27L/TNFSF7, CD28/TP44, CD80/B7-1, CD86/B7-2, A2AR, KIR (Killer-cell Immunoglobulin-like Receptor), NOX2/nicotinamide adenine dinucleotide phosphate NADPH oxidase isoform 2, SIGLEC7 (Sialic acid-binding immunoglobulin-type lectin 7)/CD328, SIGLEC9 (Sialic acid-binding immunoglobulin-type lectin 9)/CD329, CD80/B7-1, CD86/B7-2, B7-H3/CD276, VTCN1/B7-H4/B7S1/7x, VISTA (V-domain Ig suppressor of T cell activation)/B7-H5/GI24, LAG-3/CD223/Lymphocyte activation gene 3, indole amine 2,3-dioxygenase-dioxygenase/IDO, TDO/tryptophan 2,3-dioxygenase, Galectin-/LGALS9, TIGIT/VSTM3, HVEM (Herpesvirus Entry Mediator)/TNFRSF14, BTLA (B and T Lymphocyte Attenuator)/CD272, CD160, CEACAM1/CD66a, indole amine, SIRP, alpha/CD172a, CD47, CD48/SLAMF2, CD30, CD30 ligand/CD30L, TMIGD2, HHLA2, TL1A, DR3, LTOR, TNF, TNFR2 and 2B4/CD244.

In a preferred embodiment, said immune checkpoint protein is a T cell-associated checkpoint inhibitor or a non-T-cell associated checkpoint inhibitor, preferably a T cell-associated checkpoint inhibitor.

In a preferred embodiment, said immune checkpoint protein is selected from the group consisting of CD137/4-1BB, CD40/TNFRSF5, OX40/CD134, GITR/TNFRSF18, ICOS (Inducible T-cell Co-stimulator)/AILIM/CD278, CD122, A2AR (Adenosine A2A receptor), KIR, NOX2, SIGLEC7/CD328, SIGLEC9/CD329, PD-1, PD-L1, PD-L2, CTLA-4, CD80/B7-1, CD86/B7-2, B7-H3/CD276, B7-H4/B7S1/7x, VISTA/B7-H5/GI24, LAG-3/CD223/Lymphocyte activation gene 3, 2,3-dioxygenase/IDO, Galectin-/LGALS9, TIM-3/HAVCR2, and TIGIT/VSTM3. In a further preferred embodiment, said immune checkpoint protein is selected from the group consisting of PD-1, PD-L1, PD-L2, CTLA-4, B7-1, B7-2, 4-1BB, TIGIT, LAG-3, TIM3, GITR, CD40, OX40, and ICOS. In a further preferred embodiment, said immune checkpoint protein is selected from the group consisting of PD-1, PD-L1, PD-L2, CTLA-4, B7-1, B7-2, 4-1BB, TIGIT, LAG-3, TIM-3, GITR, CD40, OX40, and ICOS. In a further preferred embodiment, said immune checkpoint protein is selected from the group consisting of PD-1, PD-L1, PD-L2, CTLA-4, 4-1BB, TIGIT, LAG-3, TIM3, GITR, CD40, OX40, and ICOS. In a further preferred embodiment, said immune checkpoint protein is selected from the group consisting of PD-1, PD-L1, PD-L2, 4-1BB, GITR, CD40, and OX40. In a further preferred embodiment, said immune checkpoint protein is selected from the group consisting of PD-1, PD-L1, PD-L2, CTLA-4, 4-1BB, GITR, CD40, and OX40. In a further preferred embodiment, said immune checkpoint protein is selected from the group consisting of PD-1, PD-L1, PD-L2, 4-1BB, GITR, CD40, and OX40. In a further preferred embodiment, said immune checkpoint protein is selected from the group consisting of PD-1, PD-L1, PD-L2, 4-1BB, GITR, and OX40. In a further preferred embodiment, said immune checkpoint protein is selected from the group consisting of PD-1, PD-L1, PD-L2, 4-1BB, and OX40. In a further preferred embodiment, said immune checkpoint protein is selected from the group consisting of PD-1, PD-L1, PD-L2, 4-1BB, LAG-3, and TIM3. In a further preferred embodiment, said immune checkpoint protein is selected from the group consisting of PD-1, PD-L1, PD-L2, CTLA-4, 4-1BB, LAG-3, and TIM3. In a further preferred embodiment, said immune checkpoint protein is selected from the group consisting of PD-1, PD-L1, PD-L2, CTLA-4, 4-1BB, TIGIT, LAG-3, TIM-3, GITR, and ICOS. In a further preferred embodiment, said immune checkpoint protein is selected from the group consisting of PD-1, PD-L1, PD-L2, CTLA-4, 4-1BB, LAG-3, TIGIT, TIM-3 and GITR. In a further preferred embodiment, said immune checkpoint protein is selected from the group consisting of PD-1, CTLA-4, 4-1BB, LAG-3, TIGIT, TIM-3, GITR, and ICOS. In a further preferred embodiment, said immune checkpoint protein is selected from the group consisting of PD-1, PD-L1, PD-L2, CTLA-4, and 4-1BB. In a further preferred embodiment of the composition or kit-of-parts of the invention, said immune checkpoint protein is selected from the group consisting of 4-1n, PD-1, PD-L1, PD-L2 and CTLA-4.

In a further very preferred embodiment, said immune checkpoint protein is selected from the group consisting of PD-1, PD-L1, PD-L2, and 4-1BB. In a further preferred embodiment, said immune checkpoint protein is PD-1 or 4-1BB. In a further preferred embodiment, said immune checkpoint protein is PD-1. In another preferred embodiment, said immune checkpoint protein is PD-1, PD-L1, or PD-L2. In a further preferred embodiment, said immune checkpoint protein is PD-1, or PD-L1. In a further preferred embodiment, said immune checkpoint protein is PD-1. In a further preferred embodiment, said immune checkpoint protein is PD-L1. In a further preferred embodiment, said immune checkpoint protein is PD-L2. In a further preferred embodiment, said immune checkpoint protein is 4-1BB. In another preferred embodiment, said immune checkpoint protein is CTLA-4.

In a very preferred embodiment said immune checkpoint modulator is an antibody capable of modulating activity of an immune checkpoint protein, mentioned herein also as immunomodulatory antibody.

Preferably, said immune checkpoint modulator is an immunomodulatory agonistic or antagonistic antibody. Said immunomodulatory antibody includes agonistic antibodies of co-stimulatory checkpoint proteins and their ligands and antagonistic antibodies of inhibitory checkpoint protein and their ligands.

As used herein, the term “antibody” or “anti- . . . ” (binding to a target mentioned after the term “anti-”) to immunoglobulin molecules and portions of immunoglobulin molecules, preferably said portions contain an antigen binding site that selectively binds an antigen wherein the antigen comprises haptens, epitopes, receptors or ligands or parts thereof. The term “antibody capable of modulating an immune checkpoint protein” as used herein refers preferably to antibodies that modulate activity of checkpoint receptors, counter-receptors or bound and soluble ligands thereof. As such, the term “antibody” encompasses not only whole antibody molecules, but also antibody fragments as well as variants (including derivatives), antibody fragments, fusion proteins, and antibody mimetics, such as ankyrin repeats (DARPins), adnectins, affibodies, or engineered Kunitz-type inhibitors. The term “antibody” includes antibodies comprising two immunoglobulin heavy chains and two immunoglobulin light chains as well as a variety of forms including full length antibodies and portions thereof; including, for example, an immunoglobulin molecule, a monoclonal antibody, a chimeric antibody, a CDR-grafted antibody, a humanized antibody, Fab, Fab′, F(ab′)2, Fv, disulfide linked Fv, scFv, single domain antibody (dAb), a diabody; DNA, RNA or peptidic aptamers; affibodies; naked antibodies, antibody-drug conjugates and bi- or tri-specific antibodies, an anti-idiotypic antibody, anticalin and a functionally active epitope-binding fragment of these. In a particularly preferred embodiment, said antibody capable of modulating an immune checkpoint protein is a monoclonal antibody, a humanized antibody or a full human antibody.

As used herein, the term “affibody” refers to proteins engineered to bind to target proteins or peptides with high affinity, imitating monoclonal antibodies, and are therefore a member of the family of antibody mimetics. Preferably the affibody has a high affinity binding domain derived from protein A.

In a preferred embodiment, said immune checkpoint modulator capable of modulating the immune checkpoint protein is a monoclonal antibody, chimeric antibody, humanized antibody, human antibody, a fusion protein or a combination thereof.

In a preferred embodiment, said antibody is selected from the group consisting of anti-4-1BB, anti-CD40/TNFRSF5, anti-OX40/CD134, anti-GITR/TNFRSF18, anti-ICOS (Inducible T-cell Co-stimulator)/AILIM/CD278, anti-/B7-H2, anti-CD122, anti-A2AR (Adenosine A2A receptor), anti-KIR, anti-NOX2, anti-SIGLEC7/CD328, anti-SIGLEC9/CD329, anti-PD-1, anti-PD-L1, anti-PD-L2, anti-CTLA-4, anti-CD80/B7-1, anti-CD86/B7-2, anti-B7-H3/CD276, anti-B7-H4/B7S1/7x, anti-VISTA/B7-H5/GI24, anti-LAG-3/CD223/Lymphocyte activation gene 3, anti-2,3-dioxygenase/IDO, anti-Galectin-/LGALS9, anti-TIM-3/HAVCR2, and anti-TIGIT/VSTM3.

In a further preferred embodiment, said antibody is selected from the group consisting of anti-PD-1, anti-PD-L1, anti-PD-L2, anti-CTLA-4, anti-4-1BB, anti-LAG-3, anti-TIGIT, anti-TIM-3, anti-GITR, and anti-ICOS. In a further preferred embodiment, said antibody is selected from the group consisting of anti-PD-1, anti-PD-L1, anti-PD-L2, anti-4-1BB, anti-GITR, anti-CD40, anti-ICOS and anti-OX40. In a further preferred embodiment, said antibody is selected from the group consisting of anti-PD-1, anti-PD-L1, anti-PD-L2, anti-4-1BB, anti-GITR, anti-CD40 and anti-OX40. In a further preferred embodiment, said antibody is selected from the group consisting of anti-PD-1, anti-PD-L1, anti-PD-L2, anti-4-1BB, anti-GITR and anti-OX40. In a further preferred embodiment, said antibody is selected from the group consisting of anti-PD-1, anti-PD-L1, anti-PD-L2, anti-4-1BB, and anti-OX40. In a further preferred embodiment, said antibody is selected from the group consisting of anti-PD-1, anti-PD-L1, anti-PD-L2, anti-CTLA-4, and anti-4-1BB. In a very preferred embodiment, said antibody is selected from the group consisting of anti-PD-1, anti-PD-L1, anti-PD-L2, and anti-4-1BB. In a further preferred embodiment, said antibody is anti-PD-1 or anti-4-1BB. In a further preferred embodiment, said antibody is anti-PD-1. In another preferred embodiment, said antibody is anti-PD-1, anti-PD-L1, or anti-PD-L2. In a further preferred embodiment, said antibody is anti-PD-1, or anti-PD-L1. In a further preferred embodiment, said antibody is anti-PD-1. In a further preferred embodiment, said antibody is anti-PD-L1. In a further preferred embodiment, said antibody is anti-PD-L2. In a further preferred embodiment, said antibody is anti-4-1BB. In another preferred embodiment said antibody is an anti-CTLA-4.

In a further preferred embodiment, said at least one antibody is selected from the group consisting of (i) anti-4-1BB (also called anti-CD137 or anti-4-1BB herein), anti-CD40/TNFRSF5, anti-OX40/CD134, anti-GITR/TNFRSF18, anti-ICOS/AILIM/CD278, and anti-B7-H2 or (ii) anti-PD-1, anti-PD-L1, anti-PD-L2, anti-CTLA-4, anti-CD80/B7-1, anti-CD86/B7-2, anti-B7-H3/CD276, anti-B7-H4/B7S1/7x, anti-VISTA/B7-H5/GI24, anti-LAG-3/CD223/Lymphocyte activation gene 3, anti-Galectin-/LGALS9, anti-TIM-3/HAVCR2, and anti-TIGIT/VSTM3; or (iii) a mixture of at least one antibody of (i) and at least one antibody of (ii).

In a further preferred embodiment, said at least one antibody is selected from the group consisting of (i) anti-4-1BB, anti-GITR, anti-OX40, anti-ICOS, and anti-CD40; or (ii) anti-PD-1, anti-PD-L1, anti-PD-L2, anti-CTLA-4, anti-TIGIT, anti-LAG-3, anti-TIM3, anti-B7-H3, anti-B7-H4, anti-VISTA, and anti-CCR4; or (iii) a mixture of at least one antibody of (i) and at least one antibody of (ii). In a further preferred embodiment, said at least one antibody is selected from the group consisting of (i) anti-CD137/4-1BB; or (ii) anti-PD-1, anti-PD-L1, anti-PD-L2, anti-CTLA-4; or (iii) a mixture of at least one antibody of (i) and at least one antibody of (ii). In a further preferred embodiment, said at least one antibody is selected from the group consisting of (i) anti-CD137/4-1BB; or (ii) anti-PD-1, anti-PD-L1, anti-PD-L2; or (iii) a mixture of at least one antibody of (i) and at least one antibody of (ii).

In a further preferred embodiment, said at least one antibody is a mixture of at least one antibody selected from the group consisting of (i) anti-CD137/4-1BB; and (ii) anti-PD-1, anti-PD-L1, anti-PD-L2. In a further preferred embodiment, said at least one antibody is a mixture of anti-4-1BB and at least one antibody selected from the group consisting of (ii) anti-PD-1, anti-PD-L1, anti-PD-L2. In a further preferred embodiment, said at least one antibody is a mixture of (i) anti-4-1BB and (ii) anti-PD-1.

In another preferred embodiment, said antibody capable of modulating an immune checkpoint protein is a bispecific antibody. Preferably said bispecific antibody is capable of binding to (i) 4-1BB; and/or (ii) PD-1, PD-L1 or PD-L2. In a further preferred embodiment, said bispecific antibody is capable of binding to (i) 4-1BB; and/or (ii) PD-1, PD-L1 or PD-L2. In a further preferred embodiment, said bispecific antibody is capable of binding to (i) 4-1BB and/or (ii) PD-L1.

In another preferred embodiment, said immune checkpoint modulator is a bispecific antibody with specificity for PSMA and the immune checkpoint protein. In another preferred embodiment, said immune checkpoint modulator is a bispecific antibody capable of binding to the cancer antigen PSMA, and an immune checkpoint protein selected from the group consisting of PD-1, CTLA-4, and 4-1BB. More preferably, said immune checkpoint modulator is a bi-specific PSMA-targeted oligonucleotide, a PSMA-targeted multimeric antibody or a bispecific DARPin, each of them binds to PSMA and an immune checkpoint modulator, preferably, said immune checkpoint protein is selected from the group consisting of PD-1, CTLA-4, and 4-1BB, more preferably selected from the group consisting of PD-1, and 4-1BB.

Preferred examples of said antibody capable of modulating an immune checkpoint protein are human or humanized monoclonal anti-CTLA-4, anti-PD-1, anti-PD-L1 or anti-PD-L2 antibodies. Preferred examples of said antibody capable of modulating an immune checkpoint protein are human or humanized monoclonal anti-CTLA-4 antibodies or human or humanized monoclonal anti-PD-1 antibodies. Preferably, said human or humanized monoclonal antibody is an IgG4x antibody.

Preferred examples of said antibody capable of modulating the immune checkpoint PD-1 (anti-PD-1) are human or humanized monoclonal anti-PD-1 antibodies. Preferably, said human or humanized monoclonal anti-PD-1 antibody is an IgG4x antibody. Preferred examples of said antibody capable of modulating the immune checkpoint protein PD-1 (anti-PD-1) are human or humanized antibodies selected from the group consisting of pembrolizumab, nivolumab (known also as MDX-1106 or BMS-936558, Topalian et al., 2012. N. Eng. J. Med. 366:2443-2454, disclosed in U.S. Pat. No. 8,008,449 B2), cemiplimab, IBI308, BCD-100, PDR001, tislelizumab, camrelizumab, pidilizumab (disclosed in Rosenblatt et al., 2011, J Immunother. 34:409-18), and lambrolizumab (e.g. disclosed as hPD-109A and its humanized derivatives h409All, h409A16 and h409A17 in WO2008/156712; Hamid et al., 2013, N. Engl. J. Med. 369: 134-144) and soluble PD-1 ligands including without limitation PD-L2 Fc fusion protein (also known as B7-DC-Ig or AMP-244; disclosed in Mkrtichyan M, et al., 2012, J Immunol. 189: 2338-47). More preferred examples of said antibody capable of modulating the immune checkpoint protein PD-1 (anti-PD-1) are pembrolizumab or nivolumab. Most preferably, said immune checkpoint modulator is nivolumab.

Preferred examples of said antibody capable of modulating the immune checkpoint protein PD-L1 or PD-L2 are human or humanized anti-PD-L1 or PD-L2 monoclonal antibodies. Preferably, said human or humanized monoclonal anti-PD-L1 or -L2 antibody is an IgG4x antibody. Preferred examples of said antibody capable of modulating the immune checkpoint protein PD-L1 (anti-PD-L1) are antibodies selected from the group consisting of durvalumab, avelumab, and atezolizumab, MEDI-4736 (disclosed e.g. in WO 2011/066389 A1), MPDL328 OA (disclosed e.g. in U.S. Pat. No. 8,217,149 B2) and MIH1 (Affymetrix). More preferred examples of said antibody capable of modulating the immune checkpoint protein PD-L1 (anti-PD-L1) are antibodies selected from the group consisting of durvalumab, avelumab, and atezolizumab.

Preferred examples of said antibody capable of modulating the immune checkpoint protein CTLA-4 (anti-CTLA-4) are human or humanized anti-CTLA-4 monoclonal antibodies. Preferably, said human or humanized monoclonal anti-CTLA-4 antibody is an IgG4x antibody. Preferred examples of said antibody capable of modulating the immune checkpoint protein CTLA-4 are ipilimumab or tremelimumab, more preferably ipilimumab. Ipilimumab is a fully human CTLA-4 blocking antibody presently marketed under the name Yervoy (Bristol-Myers Squibb). A further CTLA-4 inhibitor is tremelimumab (referenced in Ribas et al., 2013, J. Clin. Oncol. 31:616-22).

A preferred example of said antibody capable of modulating the immune checkpoint protein CD27 is CDX-1127, an agonistic anti-CD27 monoclonal antibody. Preferred examples of said antibody capable of modulating the immune checkpoint protein OX40 are MEDI0562, a humanized OX40 agonist; MEDI6469, a murine OX40 agonist; and MEDI6383, an OX40 agonist. A preferred example of said antibody capable of modulating the immune checkpoint protein KIR is lirilumab, a monoclonal antibody to KIR. A preferred example of said antibody capable of modulating the immune checkpoint protein 4-1BB is urelumab. A preferred example of said antibody capable of modulating the immune checkpoint protein LAG-3 is relatlimab. A preferred example of said antibody capable of modulating the immune checkpoint protein LAG-3 is the monoclonal antibody BMS-986016.

In a further preferred embodiment, said at least one antibody capable of modulating an immune checkpoint protein is selected from the group consisting of pembrolizumab, nivolumab, cemiplimab, IBI308, BCD-100, PDR001, tislelizumab, camrelizumab, pidilizumab, lambrolizumab, h409All, h409A16, h409A17, soluble PD-1 ligands such as PD-L2 Fc fusion protein, durvalumab, avelumab, atezolizumab, MEDI-4736, MPD-L328 OA, CDX-1127, MIH1, MEDI0562, MEDI6469, MEDI6383, ipilimumab, tremelimumab, relatlimab, urelumab, anti-TIGIT antibody, anti-TIM3 antibody, anti-GITR antibody and anti-ICOS antibody. In a further preferred embodiment, said at least one antibody capable of modulating an immune checkpoint protein is selected from the group consisting of pembrolizumab, nivolumab, cemiplimab, IBI308, BMS-986016, BCD-100, PDR001, tislelizumab, camrelizumab, pidilizumab, lambrolizumab, h409All, h409A16, h409A17, soluble PD-1 ligands such as PD-L2 Fc fusion protein, durvalumab, avelumab, atezolizumab, Lirilumab, MEDI-4736, MPDL328 OA, MIH1, ipilimumab, tremelimumab, relatlimab and urelumab. In a further preferred embodiment, said at least one antibody capable of modulating an immune checkpoint protein is selected from the group consisting of pembrolizumab, nivolumab, cemiplimab, IBI308, BCD-100, PDR001, tislelizumab, camrelizumab, pidilizumab, lambrolizumab, h409All, h409A16, h409A17, soluble PD-1 ligands such as PD-L2 Fc fusion protein, durvalumab, avelumab, atezolizumab, MEDI-4736, MPDL328 OA, MIH1, ipilimumab, tremelimumab, and urelumab. In a further preferred embodiment, said at least one antibody capable of modulating an immune checkpoint protein is selected from the group consisting of pembrolizumab, nivolumab, cemiplimab, IBI308, BCD-100, PDR001, tislelizumab, camrelizumab, pidilizumab, lambrolizumab, h409All, h409A16, h409A17, soluble PD-1 ligands such as PD-L2 Fc fusion protein, durvalumab, avelumab, atezolizumab, MEDI-4736, MPDL328 OA, MIH1, tremelimumab, and urelumab. In a further preferred embodiment, said at least one antibody capable of modulating an immune checkpoint protein is selected from the group consisting of pembrolizumab, nivolumab, cemiplimab, IBI308, BCD-100, PDR001, tislelizumab, camrelizumab, pidilizumab, lambrolizumab, h409All, h409A16, h409A17, soluble PD-1 ligands such as PD-L2 Fc fusion protein, durvalumab, avelumab, atezolizumab, MEDI-4736, MPDL328 OA and MIH1.

In a preferred embodiment, said at least one modulator capable of modulating an immune checkpoint protein is (i) at least one modulator, preferably an antibody, capable of agonizing co-stimulatory immune checkpoint proteins (called herein also checkpoint activator); (ii) at least one modulator, preferably an antibody, capable of antagonizing inhibitory immune checkpoint proteins (called herein checkpoint inhibitor); or (iii) a mixture of both (i) and (ii). The term “antagonizing” as used herein refers to reduction or blocking activity. In a preferred embodiment, said at least one immune checkpoint modulator is a checkpoint inhibitor. In another preferred embodiment, said at least one immune checkpoint modulator is a checkpoint activator. In another preferred embodiment, said at least one immune checkpoint modulator, preferably at least one antibody, is an immune checkpoint activator or inhibitor. In another preferred embodiment, said at least one immune checkpoint modulator, preferably antibody, is an immune checkpoint activator and inhibitor.

Said checkpoint activator activates co-stimulatory immune checkpoint proteins. Checkpoint activators deliver (either directly or indirectly) activating signals preferably to T cells, B cells or natural killer cells by agonizing the receptors of said cell types or by agonizing counter-receptors on antigen-presenting cells (APCs). Said checkpoint inhibitor disinhibits, i.e. reduces or blocks inhibitory function of inhibitory immune checkpoint proteins. Checkpoint inhibitors deliver antagonizing signals to T cells, B cells or natural killer cells either directly by antagonizing the receptors of said cell types, agonizing of inhibitory ligands or antagonizing counter-receptors on antigen-presenting cells (APCs).

In a further preferred embodiment, said at least one immune checkpoint modulator is (i) at least one modulator capable of agonizing a co-stimulatory immune checkpoint protein, wherein said co-stimulatory immune checkpoint protein is selected from the group consisting of CD155/PVR, CD226/DNAM-1, CD137/4-1BB, CD40/TNFRSF5, OX40/CD134, CD27, CD122, HVEM/TNFRSF14, TNFSF14/LIGHT/CD258, CD70/CD27L/TNFSF7, CD28/TP44, CD30, TMIGD2, HHLA2, CD80/B7-1, CD86/B7-2, GITR/TNFRSF18, DR3, TL1A, TMIGD2, HHLA2, TL1A, DR3, LTOR, TNF, TNFR2, ICOS/AILIM/CD278, and B7-H2 or (ii) at least one modulator capable of antagonizing a co-inhibitory immune checkpoint protein, wherein said co-inhibitory immune checkpoint protein is selected from the group consisting of PD-1, PD-L1, PD-L2, CTLA-4, CD80/B7-1, CD86/B7-2, B7-H3/CD276, B7-H4/B7S1/7x, VISTA/B7-H5/GI24, HVEM/TNFRSF14, BTLA, CD160, LAG-3/CD223/Lymphocyte activation gene 3, CEACAM1/CD66a, indole amine, 2,3-dioxygenase/IDO, Galectin-/LGALS9, TIM-3/HAVCR2, 2B4/CD244, SIRP, alpha/CD172a, CD47, CD48/SLAMF2, TIGIT/VSTM3, A2AR, KIR, NOX2, SIGLEC7/CD328, SIGLEC9/CD329 and, or (iii) a mixture of both.

In a preferred embodiment of the composition or the kit-of-parts of the invention, said at least one immune checkpoint modulator is (i) at least one modulator capable of agonizing a co-stimulatory immune checkpoint protein, wherein said co-stimulatory immune checkpoint protein is selected from the group consisting of 4-1BB, CD40, OX40, GITR, ICOS or (ii) at least one modulator capable of antagonizing a co-inhibitory immune checkpoint protein, wherein said co-inhibitory immune checkpoint protein is selected from the group consisting of PD-1, PD-L1, PD-L2, CTLA-4, B7-1, B7-2, B7-H3, B7-H4, VISTA, LAG-3, Galectin-9, TIM-3, and TIGIT; or (iii) a mixture of (i) and (ii).

In a further preferred embodiment, said at least one immune checkpoint modulator is (i) at least one modulator capable of agonizing 4-1BB; or (ii) at least one modulator capable of antagonizing a co-inhibitory immune checkpoint protein, wherein said co-inhibitory immune checkpoint protein is selected from the group consisting of PD-1, PD-L1, PD-L2, CTLA-4; or (iii) a mixture of both. In a further preferred embodiment, said at least one immune checkpoint modulator is (i) at least one modulator capable of agonizing 4-1BB; or (ii) at least one modulator capable of antagonizing a co-inhibitory immune checkpoint protein, wherein said co-inhibitory immune checkpoint protein is selected from the group consisting of PD-1, PD-L1, PD-L2; or (iii) a mixture of both. In a further preferred embodiment, said at least one modulator capable of modulating an immune checkpoint protein is (i) at least one modulator capable of agonizing CD137/4-1BB; or (ii) at least one modulator capable of antagonizing a co-inhibitory immune checkpoint protein, wherein said co-inhibitory immune checkpoint protein is selected from the group consisting of PD-1, PD-L1, PD-L2, preferably PD-1; or (iii) a mixture of both. In a further preferred embodiment, said at least one immune checkpoint modulator is a mixture of (i) at least one modulator capable of agonizing CD137/4-1BB; and (ii) at least one modulator capable of antagonizing a co-inhibitory immune checkpoint protein, wherein said co-inhibitory immune checkpoint protein is selected from the group consisting of PD-1, PD-L1, PD-L2, preferably PD-1.

In a further preferred embodiment, said at least one modulator capable of modulating an immune checkpoint protein is (ii) at least one modulator capable of antagonizing a co-inhibitory immune checkpoint protein, wherein said co-inhibitory immune checkpoint protein is selected from the group consisting of PD-1, PD-L1, and PD-L2.

In a preferred embodiment, said composition or kit-of-parts of the invention comprises more than one antibody capable of modulating an immune checkpoint protein. In a preferred embodiment, said composition or kit-of-parts of the invention comprises 1, 2 or 3 antibodies capable of modulating an immune checkpoint protein. In a preferred embodiment, said composition or kit-of-parts of the invention comprises 2 or 3 antibodies capable of modulating an immune checkpoint protein. In a preferred embodiment, said composition or kit-of-parts of the invention comprises 2 antibodies capable of modulating an immune checkpoint protein. In a preferred embodiment, said composition or kit-of-parts of the invention comprises 3 antibodies capable of modulating an immune checkpoint protein.

In a preferred embodiment, said composition or kit-of-parts of the invention further comprises a chemotherapeutic agent. In a preferred embodiment, said composition or kit-of-parts of the invention is combined with radiotherapy.

In a preferred embodiment, said polyplex of the invention comprises a double stranded RNA (dsRNA) and a polymeric conjugate, wherein said polymeric conjugate comprises a polyethyleneimine (PEI), one or more polyethylene glycol (PEG) moieties, one or more linkers and one or more targeting moieties; wherein said PEI is covalently bound to one or more PEG moieties, and each of said one or more PEG moieties is linked to one of said one or more targeting moieties via one of said one or more linkers, and wherein each of said one or more targeting moieties is capable of binding to a cancer antigen.

In a preferred embodiment, said polyplex of the invention comprises a double stranded RNA (dsRNA) and a polymeric conjugate, wherein said polymeric conjugate consists of a polyethyleneimine (PEI), one or more polyethylene glycol (PEG) moieties, one or more linkers and one or more targeting moieties; wherein said PEI is covalently bound to one or more PEG moieties, and each of said one or more PEG moieties is linked to one of said one or more targeting moieties via one of said one or more linkers, and wherein each of said one or more targeting moieties is capable of binding to a cancer antigen.

In a preferred embodiment, said polyplex of the invention consists of a double stranded RNA (dsRNA) and a polymeric conjugate, wherein said polymeric conjugate consists of a polyethyleneimine (PEI), one or more polyethylene glycol (PEG) moieties, one or more linkers and one or more targeting moieties; wherein said PEI is covalently bound to one or more PEG moieties, and each of said one or more PEG moieties is linked to one of said one or more targeting moieties via one of said one or more linkers, and wherein each of said one or more targeting moieties is capable of binding to a cancer antigen.

The polyplex of the invention comprises a double stranded RNA (dsRNA). The term “dsRNA” typically and preferably refers to double stranded ribonucleotide polymers of any length in which one or more ribonucleotides can be chemical analogues or modified derivatives of a corresponding naturally occurring ribonucleotide. The term “dsRNA” typically and preferably also includes mismatched dsRNA.

In a preferred embodiment, said dsRNA is polyinosinic-polycytidylic acid double stranded RNA (polyIC or pIC). PolyIC is a double-stranded RNA with one strand being a polymer of inosinic acid, the other a polymer of cytidylic acid.

The polyIC of the polyplex for use according to the invention may be composed of dsRNA, wherein each strand consists of at least 22, preferably at least 45 ribonucleotides. In a certain embodiment, each strand consists of 20 to 8000 ribonucleotides.

The term “molecular weight” as used herein and, in particular when referring to polymers such as polyIC, PEI and PEG, refers to average molecular weight, preferably to weight average molecular weight.

PolyIC is bound to the polymeric conjugate via non-covalent or covalent bonds, wherein non-covalent binding is preferred. In a preferred embodiment, said polyIC is non-covalently bound to PEI, preferably by ionic bonds.

The polyplex according to the invention comprises a polymeric conjugate, wherein said polymeric conjugate comprises polyethyleneimine (PEI) which is a polycation with the capacity to condense and associate non-covalently with nucleic acid molecules due to the polyanionic nature of the latter.

In a preferred embodiment, said polyethyleneimine (PEI) is linear polyethyleneimine (LPEI). In a preferred embodiment, LPEI includes a hydroxyl group located at one or either end of LPEI. Preferably, said hydroxyl group is instead of the terminal —NH₂ group or terminal-NHCH₃ of LPEI.

In a preferred embodiment of the invention, PEI or preferably LPEI has a molecular weight from about 10-30 kDa. In a preferred embodiment of the invention, PEI or preferably LPEI has a molecular weight from about 15-25 kDa (PEI₁₅₋₂₅ₖ//LPEI₁₅₋₂₅ₖ).

In a preferred embodiment, said PEI or preferably LPEI has a low dispersity. Preferably, the polydispersity index PDI of PEI or LPEI is about 1 (SPECS NMT 1.1).

In a preferred embodiment, said one or more PEG moieties each independently forms —NH—CO— bond with said PEI or preferably LPEI.

The polyplex for use according to the invention includes one or more polyethylene glycol (PEG) moieties. PEG moieties according to the invention are also known as polyethylene oxide (PEO) or polyoxyethylene (POE) moieties, depending on its molecular weight. As used herein the term “polyethylene glycol moiety” (PEG moiety) typically and preferably refers to a PEG moiety comprising two functionalities located on either end of polyethylene glycol (PEG). Said functionalities are capable of reacting with either said PEI or preferably LPEI or said targeting moiety.

In a preferred embodiment, said PEG moiety is linear or branched. In another preferred embodiment, said PEG moiety is branched. In a further preferred embodiment, said PEG moiety is linear.

In one embodiment of the invention, each of said at least one PEG moiety has a molecular weight from 1 kD or more. In another embodiment, each of said at least one PEG moiety has a molecular weight from about 0.3-8 kDa, preferably about 0.5-5 kDa, more preferably, 1-3 kDa (PEG₁₋₃ₖ). Said molecular weight corresponds to average molecular weight.

In a preferred embodiment, said PEI has a molecular weight of about 10-30 kDa, and said at least one PEG moiety has a molecular weight of about 0.3-8 kDa

In a preferred embodiment, PEI is covalently linked to one to five PEG moieties, preferably PEI is covalently linked to one to three PEG moieties. In a preferred embodiment, LPEI is covalently linked to one to five PEG moieties, wherein preferably LPEI is covalently linked to one to three PEG moieties. In a preferred embodiment, PEI₁₅₋₂₅ₖ, is covalently linked to one to five PEG₁₋₃ₖ, moieties, wherein preferably PEI₁₅₋₂₅ₖ, is covalently linked to one to three PEG₁₋₃ₖ, preferably PEG₂ₖ, moieties. In another preferred embodiment, said PEI of the polyplex is covalently bound to one, two or three PEG moieties. Preferably, said PEI of the polyplex is covalently bound to one or three PEG moieties. In another more preferred embodiment, said PEI of the polyplex is LPEI covalently bound to one, two or three PEG moieties. More preferably, said PEI of the polyplex is LPEI covalently bound to one or three PEG moieties.

As used herein, the term “PEI [ . . . ] covalently linked to one PEG moiety” (used interchangeably herein with “PEI-PEG 1:1”) or “LPEI [ . . . ] covalently linked to one PEG moiety” (used interchangeably herein with “LPEI-PEG 1:1”) refers to the molar ratio of PEI to PEG or LPEI to PEG, wherein PEI-PEG 1:1 or LPEI-PEG 1:1 typically and preferably means that approximately one mole PEG per one mole PEI or LPEI is included in the polymeric conjugate. As used herein, the term “PEI [ . . . ] covalently linked to three PEG moieties” (used interchangeably herein with “PEI-PEG 1:3”) or the term “LPEI [ . . . ] covalently linked to three PEG moieties” (used interchangeably herein with “LPEI-PEG 1:3”) typically and preferably means that approximately three moles PEG per one mole PEI or LPEI are included in the polymeric conjugate. The values are preferably determined by ¹H-NMR analysis. The relative integral values of the hydrogen atoms on PEG (—CH₂—CH₂—O—) and the integral values of the hydrogen atoms on PEI or LPEI (—CH₂—CH₂—NH—) are preferably used for determining the values via ¹H-NMR. The term “approximately” herein refers preferably to a deviation of about 0%-10%, more preferably about 0%-5%, again more preferably about 0%-2%.

In a preferred embodiment, said dsRNA is polyIC and said PEI is covalently linked to one to three PEG moieties. In a preferred embodiment, said dsRNA is polyIC and said PEI is covalently linked to one, two or three PEG moieties. In a more preferred embodiment, said dsRNA is polyIC and said PEI is LPEI covalently linked to one to three PEG moieties. In a more preferred embodiment, said dsRNA is polyIC and said PEI is LPEI covalently linked to one, two or three PEG moieties.

In a preferred embodiment, said dsRNA is polyIC and said PEI is covalently linked to one, two or three PEG moieties. In a more preferred embodiment, said dsRNA is polyIC and said PEI is LPEI covalently linked to one, two or three PEG moieties. In a more preferred embodiment, said dsRNA is polyIC and said PEI is LPEI covalently linked to one, two or three PEG_{0.3-8k} moieties.

In a preferred embodiment, said dsRNA is polyIC and said PEI is covalently linked to one PEG moiety (PEI-PEG 1:1). In a more preferred embodiment, said dsRNA is polyIC and said PEI is LPEI covalently linked to one PEG moiety (LPEI-PEG 1:1).

In a preferred embodiment, said dsRNA is polyIC and said PEI is covalently linked to three PEG moieties. In a more preferred embodiment, said dsRNA is polyIC and said PEI is LPEI covalently linked to one PEG moiety.

In a preferred embodiment, said dsRNA is polyIC and said LPEI is covalently linked to one or three PEG_{0.3-8k} moieties.

The term “cancer antigen” as used herein (synonymously used herein with tumor antigen or tumor marker) refers to an antigenic substance produced in tumor cells or presented on tumor cells which triggers an immune response in the host. Said cancer antigen is PSMA.

In another preferred embodiment, said dsRNA is polyIC. In another preferred embodiment, said dsRNA is polyIC and said PEI is LPEI covalently linked to one to three PEG moieties and said cancer antigen is PSMA.

In another preferred embodiment, said dsRNA is polyIC and said PEI is LPEI covalently linked to one to three PEG moieties and said targeting moiety is DUPA moiety or DUPA derivative.

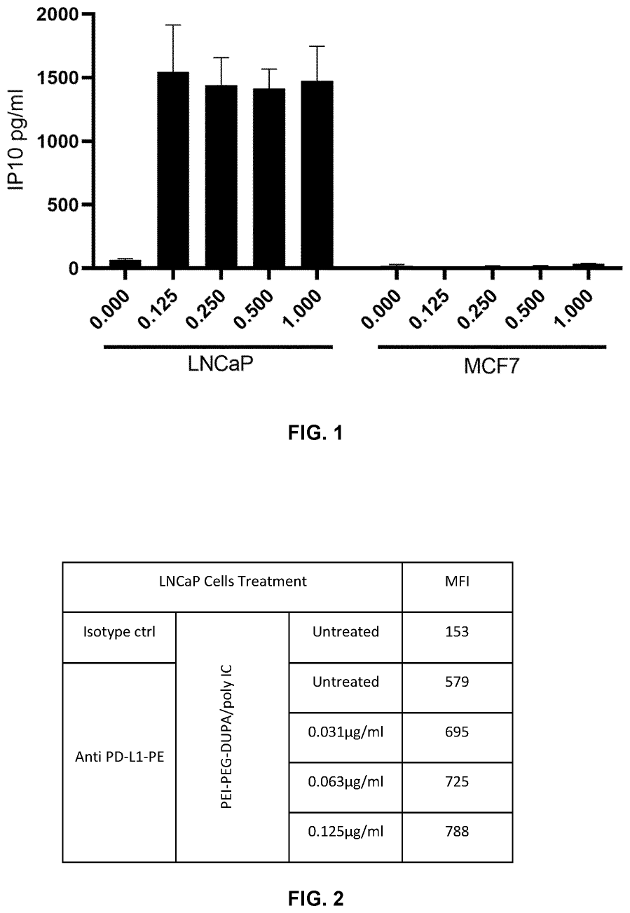

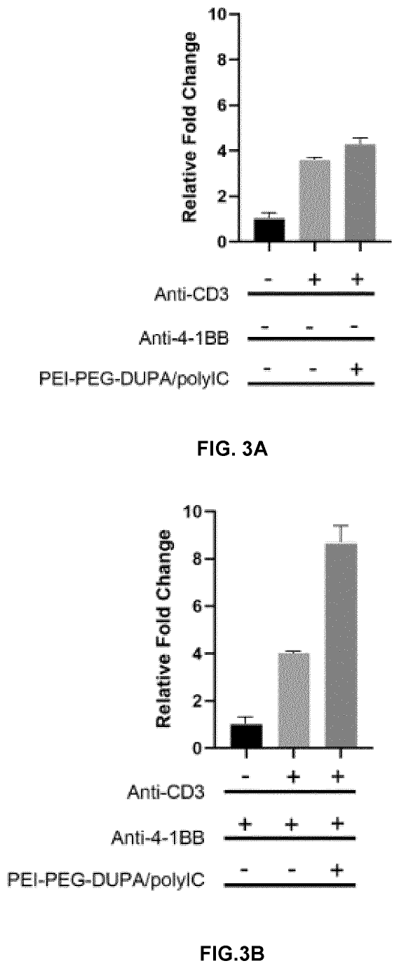

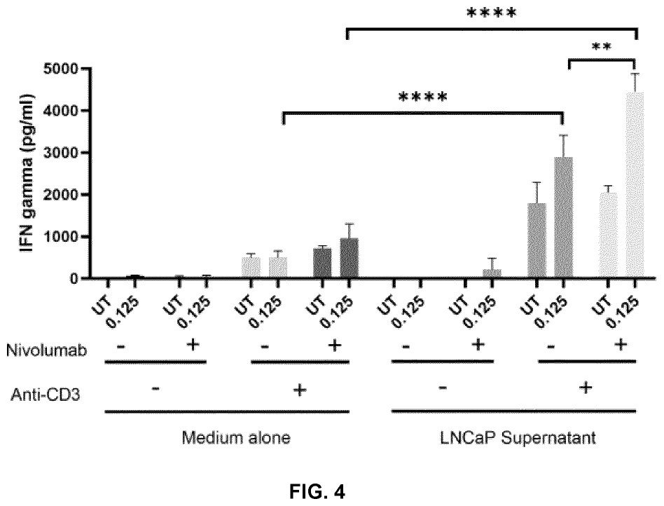

It is hereby of particular note that the vector polyIC/PEI-PEG-DUPA (pIC/PPD) comprising PEI-PEG tethered to the DUPA moiety selectively delivered polyIC into PSMA-expressing cancer cells inducing apoptosis, cytokine secretion, and the recruitment of human peripheral blood mononuclear cells (PBMCs). In particular, pIC/PPD led to the production of IFN-β, IP-10, and RANTES, to chemotaxis, and to PBMC activation which was evident from strong expression of IL-2 and led to the secretion of high levels of TNF-α and IFN-γ (Langut et al., PNAS, Dec. 26, 2017, vol. 114, no. 52, and FIG. 1, Example 1 for IP-10 release). Combination of PEI-PEG-DUPA/polyIC with Nivolumab or anti-4-1BB antibodies led to a further significant increase in cytokine IFN-γ release in PBMCs as compared to treatment with PEI-PEG-DUPA/polyIC, Nivolumab or anti-4-1BB antibodies alone (see herein FIGS. 3 and 4, Examples 2 and 3).

Therefore, the above clearly confirms and supports that the inventive compositions and kit-of-parts comprising a polyplex and at least one antibody capable of modulating an immune checkpoint protein are able to lead to increased activation of the immune system and more potent antitumor activity. This is in particular the case for the preferred inventive compositions comprising polyplexes, wherein said cancer antigen is PSMA and said one or more targeting moieties are a DUPA moiety or DUPA derivative, and wherein said antibody is capable of modulating an immune checkpoint protein, preferably an anti-4-1BB (mentioned herein also as anti-CD137), anti-PD-1, anti-PD-L1 or anti-PD-L2 antibody.

In another preferred embodiment, said dsRNA is polyIC and said PEI is LPEI₁₅₋₂₅ₖ, covalently linked to one to three PEG moieties and said targeting moiety is the DUPA moiety or DUPA derivative. In another preferred embodiment, said dsRNA is polyIC and said PEI is LPEI₁₅₋₂₅ₖ, covalently linked to one or three PEG moieties and said targeting moiety is the DUPA moiety or DUPA derivative.

In another preferred embodiment, said dsRNA is polyIC and said PEI is LPEI covalently linked to one, two or three PEG_{0.3-8k}, preferably PEG₁₋₃ₖ, further preferably PEG₂ₖ moieties and said targeting moiety is the DUPA moiety or DUPA derivative. In another preferred embodiment, said dsRNA is polyIC and said PEI is LPEI covalently linked to one or three PEG_{0.3-8k}, preferably PEG₁₋₃ₖ, further preferably PEG₂ₖ moieties and said targeting moiety is the DUPA moiety or DUPA derivative.

In another preferred embodiment, said dsRNA is polyIC and said PEI is LPEI covalently linked to one or three PEG_{0.3-8k}, preferably PEG₁₋₃ₖ, further preferably PEG₂ₖ moieties and said targeting moiety is the DUPA moiety or DUPA derivative, and said immune checkpoint protein is selected from the group consisting of PD-1, PD-L1, PD-L2, CTLA-4, 4-1BB. In another preferred embodiment, said dsRNA is polyIC and said PEI is LPEI covalently linked to one or three PEG_{0.3-8k}, preferably PEG₁₋₃ₖ, further preferably PEG₂ₖ moieties and said targeting moiety is the DUPA moiety or DUPA derivative, and said immune checkpoint protein is selected from the group consisting of PD-1, PD-L1, PD-L2, 4-1BB. In another preferred embodiment, said dsRNA is polyIC and said PEI is LPEI covalently linked to one or three PEG_{0.3-8k}, preferably PEG₁₋₃ₖ, further preferably PEG₂ₖ moieties and said targeting moiety is the DUPA moiety or DUPA derivative, and said immune checkpoint protein is selected from the group consisting of PD-1, PD-L1, PD-L2, and 4-1BB. In another preferred embodiment, said dsRNA is polyIC and said PEI is LPEI covalently linked to one or three PEG_{0.3-8k}, preferably PEG₁₋₃ₖ, further preferably PEG₂ₖ moieties and said targeting moiety is the DUPA moiety or DUPA derivative, and said immune checkpoint protein is PD-1 or 4-1BB.

In another preferred embodiment, said dsRNA is polyIC, said PEI is LPEI covalently linked to one or three PEG moieties, said targeting moiety is a PSMA targeting moiety, preferably the DUPA moiety or DUPA derivative, and said immune checkpoint protein is selected from the group consisting of PD-1, PD-L1, PD-L2, CTLA-4, B7-1, B7-2, 4-1BB, TIGIT, LAG-3, TIM3, B7-H3, B7-H4, VISTA, CCR4, GITR, OX40, ICOS, and CD40.

In another preferred embodiment, said dsRNA is polyIC, said PEI is LPEI covalently linked to one, two or three PEG moieties, said targeting moiety is a PSMA targeting moiety, preferably the DUPA moiety or DUPA derivative, and said immune checkpoint protein is selected from the group consisting of PD-1, PD-L1, PD-L2, CTLA-4, 4-1BB, TIGIT, LAG-3, TIM3, B7-H3, B7-H4, VISTA, CCR4, GITR, OX40, ICOS, and CD40. In another preferred embodiment, said dsRNA is polyIC, said PEI is LPEI covalently linked to one or three PEG moieties, said targeting moiety is a PSMA targeting moiety, preferably the DUPA moiety or DUPA derivative, and said immune checkpoint protein is selected from the group consisting of PD-1, PD-L1, PD-L2, CTLA-4, and 4-1BB. In another preferred embodiment, said dsRNA is polyIC, said PEI is LPEI covalently linked to one to three PEG moieties, said targeting moiety is a PSMA targeting moiety, preferably the DUPA moiety or DUPA derivative, and said immune checkpoint protein is selected from the group consisting of PD-1, PD-L1, PD-L2, and 4-1BB. In another preferred embodiment, said dsRNA is polyIC, said PEI is LPEI covalently linked to one or three PEG moieties, said targeting moiety is a PSMA targeting moiety, preferably the DUPA moiety or DUPA derivative, and said immune checkpoint protein is selected from the group consisting of PD-1, PD-L1, PD-L2, and 4-1BB. In another preferred embodiment, said dsRNA is polyIC, said PEI is LPEI covalently linked to one or three PEG moieties, said targeting moiety is a PSMA targeting moiety, preferably the DUPA moiety or DUPA derivative, and said immune checkpoint protein is PD-1 or 4-1BB.

In another preferred embodiment, said dsRNA is polyIC, said targeting moiety is a PSMA targeting moiety, preferably the DUPA moiety or DUPA derivative, and said immune checkpoint protein is selected from the group consisting of PD-1, PD-L1, PD-L2, 4-1BB, CTLA-4, GITR, CD40, OX40, and ICOS. In another preferred embodiment, said dsRNA is polyIC, said targeting moiety is a PSMA targeting moiety, preferably the DUPA moiety or DUPA derivative, and said immune checkpoint protein is selected from the group consisting of PD-1, PD-L1, PD-L2, and 4-1BB. In another preferred embodiment, said dsRNA is polyIC, said targeting moiety is a PSMA targeting moiety, preferably the DUPA moiety or DUPA derivative, and said immune checkpoint protein is selected from the group consisting of PD-1 and 4-1BB.

In another preferred embodiment, said dsRNA is polyIC, said PEI is LPEI covalently linked to one, two or three PEG moieties, said targeting moiety is a PSMA targeting moiety, preferably the DUPA moiety or DUPA derivative, and said immune checkpoint protein is selected from the group consisting of PD-1, PD-L1, PD-L2, CTLA-4, and 4-1BB. In another preferred embodiment, said dsRNA is polyIC, said PEI is LPEI covalently linked to one or three PEG moieties, said targeting moiety is a PSMA targeting moiety, preferably the DUPA moiety or DUPA derivative, and said immune checkpoint protein is selected from the group consisting of PD-1, PD-L1, PD-L2, CTLA-4, and 4-1BB. In another preferred embodiment, said dsRNA is polyIC, said PEI is LPEI covalently linked to one, two or three PEG moieties, said targeting moiety is a PSMA targeting moiety, preferably the DUPA moiety or DUPA derivative, and said immune checkpoint protein is selected from the group consisting of PD-1, PD-L1, PD-L2, and 4-1BB. In another preferred embodiment, said dsRNA is polyIC, said PEI is LPEI covalently linked to one, two or three PEG moieties and said targeting moiety is a PSMA targeting moiety, preferably the DUPA moiety or DUPA derivative, and said immune checkpoint protein is PD-1 and/or 4-1BB, preferably PD-1 or 4-1BB. In another preferred embodiment, said dsRNA is polyIC, said PEI is LPEI covalently linked to one, two or three PEG moieties and said targeting moiety is a PSMA targeting moiety, preferably the DUPA moiety or DUPA derivative, and said immune checkpoint protein is PD-1, PD-L1, or PD-L2. In another preferred embodiment, said dsRNA is polyIC, said PEI is LPEI covalently linked to one, two or three PEG moieties, said targeting moiety is a PSMA targeting moiety, preferably the DUPA moiety or DUPA derivative, and said immune checkpoint protein is PD-1. In another preferred embodiment, said dsRNA is polyIC, said PEI is LPEI covalently linked to one, two or three PEG moieties and said targeting moiety is a PSMA targeting moiety, preferably the DUPA moiety or DUPA derivative, and said immune checkpoint protein is 4-1BB.

In another preferred embodiment, said dsRNA is polyIC, said PEI is LPEI covalently linked to one, two or three PEG moieties, said targeting moiety is a PSMA targeting moiety, preferably the DUPA moiety or DUPA derivative, and said immune checkpoint protein modulating antibody is selected from the group consisting of anti-PD-1, anti-PD-L1, anti-PD-L2, anti-CTLA-4, and anti-4-1BB. In another preferred embodiment, said dsRNA is polyIC, said PEI is LPEI covalently linked to one or three PEG moieties, said targeting moiety is a PSMA targeting moiety, preferably the DUPA moiety or DUPA derivative, and said immune checkpoint protein modulating antibody is selected from the group consisting of anti-PD-1, anti-PD-L1, anti-PD-L2, anti-CTLA-4, and anti-4-1BB. In another preferred embodiment, said dsRNA is polyIC, said PEI is LPEI covalently linked to one, two or three PEG moieties, said targeting moiety is a PSMA targeting moiety, preferably the DUPA moiety or DUPA derivative, and said immune checkpoint protein modulating antibody is selected from the group consisting of anti-PD-1, anti-PD-L1, anti-PD-L2, and anti-4-1BB. In another preferred embodiment, said dsRNA is polyIC, said PEI is LPEI covalently linked to one, two or three PEG moieties and said targeting moiety is a PSMA targeting moiety, preferably the DUPA moiety or DUPA derivative, and said immune checkpoint protein modulating antibody is anti-PD-1 or anti-4-1BB. In another preferred embodiment, said dsRNA is polyIC, said PEI is LPEI covalently linked to one, two or three PEG moieties and said targeting moiety is a PSMA targeting moiety, preferably the DUPA moiety or DUPA derivative, and said immune checkpoint protein modulating antibody is anti-PD-1, anti-PD-L1, or anti-PD-L2. In another preferred embodiment, said dsRNA is polyIC, said PEI is LPEI covalently linked to one, two or three PEG moieties, said targeting moiety is a PSMA targeting moiety, preferably the DUPA moiety or DUPA derivative, and said immune checkpoint protein modulating antibody is anti-PD-1. In another preferred embodiment, said dsRNA is polyIC, said PEI is LPEI covalently linked to one, two or three PEG moieties and said targeting moiety is a PSMA targeting moiety, preferably the DUPA moiety or DUPA derivative, and said immune checkpoint protein modulating antibody is anti-4-1BB.

In another preferred embodiment, said dsRNA is polyIC, said PEI is covalently linked to one, two or three PEG_{0.3-8k}, said targeting moiety is a PSMA targeting moiety, preferably DUPA moiety or DUPA derivative, and said immune checkpoint protein is selected from the group consisting of PD-1, PD-L1, PD-L2, CTLA-4, B7-1, B7-2, 4-1BB, TIGIT, LAG-3, TIM3, B7-H3, B7-H4, VISTA, CCR4, GITR, OX40, ICOS, and CD40.

In another preferred embodiment, said dsRNA is polyIC, said PEI is covalently linked to one, two or three PEG_{0.3-8k} moieties, said targeting moiety is a PSMA targeting moiety, preferably DUPA moiety or DUPA derivative, and said immune checkpoint protein is selected from the group consisting of PD-1, PD-L1, PD-L2, CTLA-4, 4-1BB, GITR, CD40, OX40, and ICOS. In another preferred embodiment, said dsRNA is polyIC, said PEI is covalently linked to one, two or three PEG_{0.3-8k} moieties, said targeting moiety is a PSMA targeting moiety, preferably DUPA moiety or DUPA derivative, and said immune checkpoint protein is selected from the group consisting of PD-1, PD-L1, PD-L2, CTLA-4, and 4-1BB. In another preferred embodiment, said dsRNA is polyIC, said PEI is covalently linked to one or three PEG_{0.3-8k} moieties, said targeting moiety is a PSMA targeting moiety, preferably DUPA moiety or DUPA derivative, and said immune checkpoint protein is selected from the group consisting of PD-1, PD-L1, PD-L2, CTLA-4, and 4-1BB. In another preferred embodiment, said dsRNA is polyIC, said PEI is covalently linked to one, two or three PEG_{0.3-8k} moieties, said targeting moiety is a PSMA targeting moiety, preferably DUPA moiety or DUPA derivative, and said immune checkpoint protein is selected from the group consisting of PD-1, PD-L1, PD-L2, and 4-1BB. In another preferred embodiment, said dsRNA is polyIC, said PEI is covalently linked to one, two or three PEG_{0.3-8k} moieties and said targeting moiety is a PSMA targeting moiety, preferably DUPA moiety or DUPA derivative, and said immune checkpoint protein is PD-1 or 4-1BB. In another preferred embodiment, said dsRNA is polyIC, said PEI is covalently linked to one, two or three PEG_{0.3-8k} moieties and said targeting moiety is a PSMA targeting moiety, preferably DUPA moiety or DUPA derivative, and said immune checkpoint protein is PD-1, PD-L1, or PD-L2. In another preferred embodiment, said dsRNA is polyIC, said PEI is covalently linked to one, two or three PEG_{0.3-8k}, said targeting moiety is a PSMA targeting moiety, preferably DUPA moiety or DUPA derivative, and said immune checkpoint protein is PD-1. In another preferred embodiment, said dsRNA is polyIC, said PEI is covalently linked to one, two or three PEG_{0.3-8k} moieties and said targeting moiety is a PSMA targeting moiety, preferably DUPA moiety or DUPA derivative, and said immune checkpoint protein is 4-1BB.

In a preferred embodiment, said composition or kits-of-parts of the invention do not include, i.e. exclude a cancer vaccine or a tumor associated antigen. In a preferred embodiment, said composition or kits-of-parts of the invention includes a cancer vaccine or a tumor associated antigen.

In the polyplex of the invention, said one or more PEG moieties are linked to one of said one or more targeting moieties. In a preferred embodiment, said one or more PEG moieties are directly linked to one of said one or more targeting moieties or linked to one of said one or more targeting moieties via one of said one or more linkers.

In a further preferred embodiment, said one or more targeting moieties comprises or preferably consists of a DUPA derivative (HOOC(CH₂)₂—CH(COOH)—NH—CO—NH—CH(COOH)—CH₂—) or DUPA moiety (HOOC(CH₂)₂—CH(COOH)—NH—CO—NH—CH(COOH)—(CH₂)₂—CO—), wherein said one or more PEG moieties are directly linked to one of said one or more targeting moieties or linked to one of said one or more targeting moieties via one of said one or more linkers.

In a further very preferred embodiment, said one or more targeting moieties consists of a DUPA moiety (HOOC(CH₂)₂—CH(COOH)—NH—CO—NH—CH(COOH)—(CH₂)₂—CO—), wherein said one or more PEG moieties are directly linked to one of said one or more targeting moieties or linked to one of said one or more targeting moieties via one of said one or more linkers.

In a further very preferred embodiment, said one or more targeting moieties consists of a DUPA moiety (HOOC(CH₂)₂—CH(COOH)—NH—CO—NH—CH(COOH)—(CH₂)₂—CO—), wherein said one or more PEG moieties are linked to one of said one or more targeting moieties via one of said one or more linkers.

In a further preferred embodiment, said linker is selected from —CO—R2-RX—R3 or a peptide moiety consisting of 3 to 7 amino acid residues, wherein R2 is selected from (C₁-C₇)alkylene, (C₂-C₈)alkenylene, (C₂-C₈) alkynylene, (C₆-C₁₀)arylene-diyl, or heteroarylenediyl; RX is absent or —S—; R3 is absent or of the formula

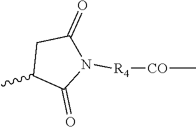

wherein R4 is selected from (C₁-C₈)alkylene, (C₂-C₈)allkenylene, (C₂-C₈) alkynylene(C₁-C₈)alkylene —(C₃-C₈)cycloallkylene, (C₂-C₈)allkenylene-(C₃-C₈)cycloallkylene, (C₂-C₈)alkynylene-(C₃-C₈)cycloallkylene, (C₆-C₁₀)arylene-diyl, heteroarylenediyl, (C₁-C₈)alkylene-(C₆-C₁₀)arylene-diyl, or (C₁-C₈)alkylene-heteroarylenediyl; wherein each one of said (C₁-C₈)allkylene, (C₂-C₈)allkenylene, or (C₂-C₈) alkynylene is optionally substituted by one or more groups each independently selected from halogen, —COR5, —COOR5, —OCOOR5, —OCON(R5)₂, —CN, —NO₂, —SR5, —OR5, —N(R5)₂, —CON(R5)₂, —SO₂R5, —SO₃H, —S(═O)R5, (C₆-C₁₀)aryl, (C₁-C₄)allkylene-(C₆-C₁₀)aryl, heteroaryl, or (C₁-C₄)allkylene-heteroaryl, and further optionally interrupted by one or more identical or different heteroatoms selected from S, O or N, and/or at least one group each independently selected from —NH—CO—, —CO—NH—, —N(R5)-, —N(C₆-C₁₀)aryl-, (C₆-C₁₀)arylene-diyl, or heteroarylenediyl; and R5 is H or (C₁-C₈)alkyl.

In a further preferred embodiment, said polymeric conjugate is of formula (i)-(iv):

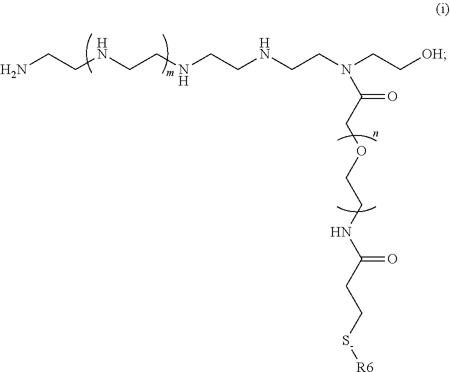

wherein R6 is

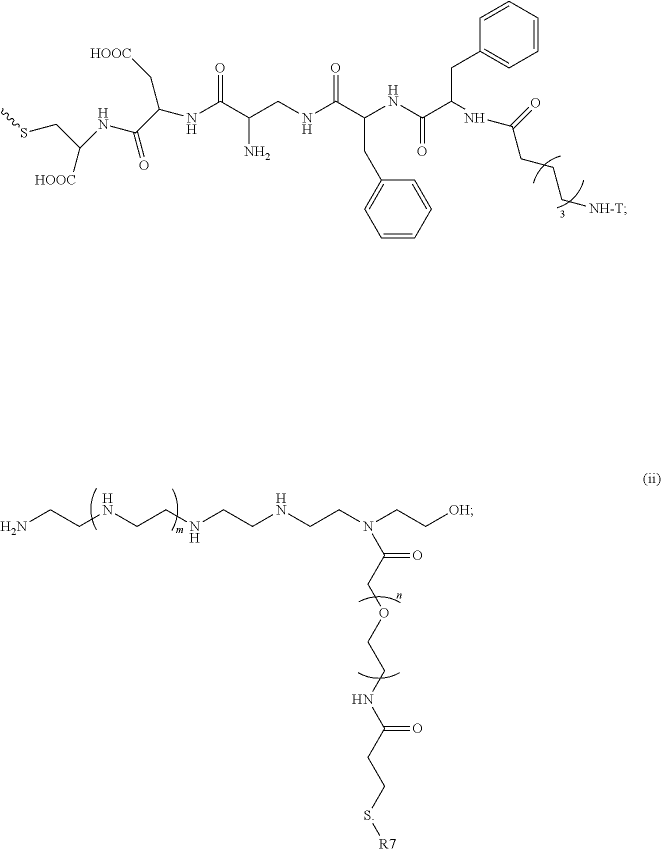

wherein R7 is

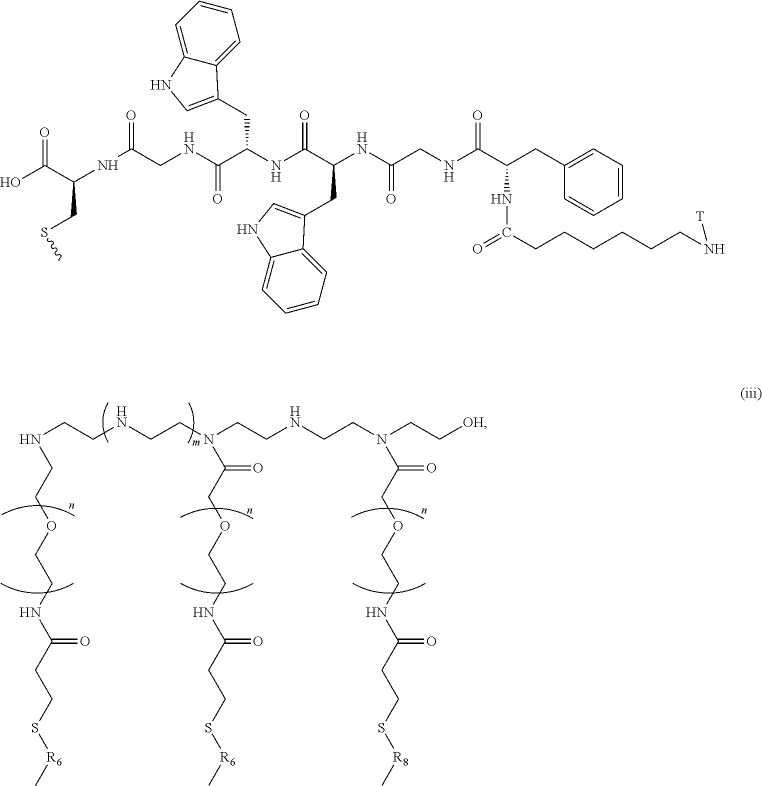

wherein R6 is

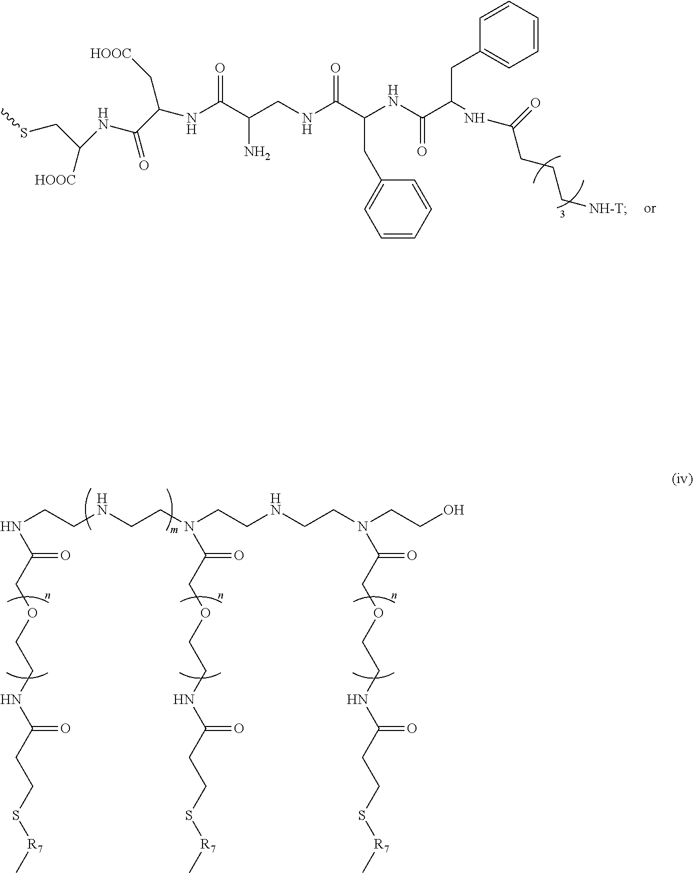

wherein R7 is

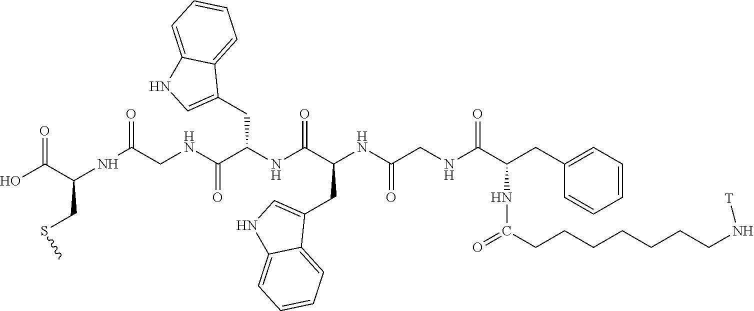

wherein said T represents said targeting moiety, n corresponds to a molecular weight of 0.3-8 kD, preferably 0.5-5 kD, more preferably 1-3 kD, most preferably 2 kD; and m corresponds to a molecular weight of 10-30 kD, preferably 15-25 kD, further preferably 22 kD.

In a further preferred embodiment, said targeting moiety is HOOC(CH₂)₂—CH(COOH)—NH—CO—NH—CH(COOH)—(CH₂)₂—CO— (DUPA moiety), and said polymeric conjugate is of the formula (i), (ii), (iii) or (iv). In a further preferred embodiment, said polymeric conjugate is of formula (i) or (iii), wherein R6 is SEQ ID NO: 1 (—(NH—(CH₂)₇—CO)-Phe-Phe-(NH—CH₂—CH(NH₂)—CO)-Asp-Cys-), and wherein T represents the targeting moiety HOOC(CH₂)₂—CH(COOH)—NH—CO—NH—CH(COOH)—(CH₂)₂—CO— (DUPA moiety). In a further preferred embodiment, said polymeric conjugate is of formula (ii) or (iv), wherein R7 is SEQ ID NO: 2 (—(NH—(CH₂)₇—CO)-Phe-Gly-Trp-Trp-Gly-Cys-), and wherein T represents the targeting moiety HOOC(CH₂)₂—CH(COOH)—NH—CO—NH—CH(COOH)—(CH₂)₂—CO— (DUPA moiety).

In another preferred embodiment, said dsRNA is polyIC, said targeting moiety is a PSMA binding targeting moiety, preferably DUPA moiety, and said immune checkpoint protein is selected from the group consisting of PD-1, PD-L1, PD-L2, CTLA-4, B7-1, B7-2, 4-1BB, TIGIT, LAG-3, TIM3, B7-H3, B7-H4, VISTA, CCR4, GITR, OX40, ICOS, and CD40, and wherein said polymeric conjugate is of the formula (i), (ii), (iii) or (iv).

In another preferred embodiment, said dsRNA is polyIC, said targeting moiety is PSMA binding targeting moiety, preferably DUPA moiety, and said immune checkpoint protein is selected from the group consisting of PD-1, PD-L1, PD-L2, CTLA-4, B7-1, B7-2, 4-1BB, TIGIT, LAG-3, TIM-3, GITR, CD40, OX40, and ICOS, and wherein said polymeric conjugate is of the formula (i), (ii), (iii) or (iv).

In another preferred embodiment, said dsRNA is polyIC, said targeting moiety is PSMA binding targeting moiety, preferably DUPA moiety or DUPA derivative, and said immune checkpoint protein is selected from the group consisting of PD-1, PD-L1, PD-L2, CTLA-4, and 4-1BB. In another preferred embodiment, said dsRNA is polyIC, said targeting moiety is PSMA binding targeting moiety, preferably DUPA moiety, and said immune checkpoint protein is selected from the group consisting of PD-1, PD-L1, PD-L2, CTLA-4, and 4-1BB, and wherein said polymeric conjugate is of the formula (i), (ii), (iii) or (iv). In another preferred embodiment, said dsRNA is polyIC, said targeting moiety is PSMA binding targeting moiety, preferably DUPA moiety, and said immune checkpoint protein is selected from the group consisting of PD-1, PD-L1, PD-L2, and 4-1BB, and wherein said polymeric conjugate is of the formula (i), (ii), (iii) or (iv). In another preferred embodiment, said dsRNA is polyIC, said targeting moiety is PSMA binding targeting moiety, preferably DUPA moiety, and said immune checkpoint protein is PD-1 or 4-1BB, and wherein said polymeric conjugate is of the formula (i), (ii), (iii) or (iv). In another preferred embodiment, said dsRNA is polyIC, said targeting moiety is PSMA binding targeting moiety, preferably DUPA moiety, and said immune checkpoint protein is PD-1, PD-L1, or PD-L2, and wherein said polymeric conjugate is of the formula (i), (ii), (iii) or (iv). In another preferred embodiment, said dsRNA is polyIC, said targeting moiety is a PSMA binding targeting moiety, preferably DUPA moiety, and said immune checkpoint protein is PD-1, and wherein said polymeric conjugate is of the formula (i), (ii), (iii) or (iv). In another preferred embodiment, said dsRNA is polyIC, said targeting moiety is a PSMA binding targeting moiety, preferably DUPA moiety, and said immune checkpoint protein is 4-1BB, and wherein said polymeric conjugate is of the formula (i), (ii), (iii) or (iv).

In another preferred embodiment, said dsRNA is polyIC, said polymeric conjugate is of the formula (ii) or (iv), said targeting moiety is a PSMA binding targeting moiety, preferably DUPA moiety, and said at least one antibody capable of modulating an immune checkpoint protein is selected from the group consisting of 4-1BB, PD-1, PD-L1, PD-L2, and CTLA-4. In another preferred embodiment, said dsRNA is polyIC, said polymeric conjugate is of the formula (ii) or (iv), said targeting moiety is a PSMA binding targeting moiety, preferably DUPA moiety, and said at least one antibody capable of modulating an immune checkpoint protein is selected from the group consisting of 4-1BB, PD-1, PD-L1, and PD-L2. In another preferred embodiment, said dsRNA is polyIC, said polymeric conjugate is of the formula (ii) or (iv), said targeting moiety is a PSMA binding targeting moiety, preferably DUPA moiety, and said at least one antibody capable of modulating an immune checkpoint protein is 4-1BB or PD-1. In another preferred embodiment, said dsRNA is polyIC, said polymeric conjugate is of the formula (ii) or (iv), said targeting moiety is a PSMA binding targeting moiety, preferably DUPA moiety, and said at least one antibody capable of modulating an immune checkpoint protein is 4-1BB. In another preferred embodiment, said dsRNA is polyIC, said polymeric conjugate is of the formula (ii) or (iv), said targeting moiety is a PSMA binding targeting moiety, preferably DUPA moiety, and said at least one antibody capable of modulating an immune checkpoint protein is PD-1.

The polyplex of the invention comprises one or more targeting moieties. Said targeting moiety may be a native, natural or modified ligand or a paralog thereof, or a non-native ligand such as an antibody, a single-chain variable fragment (scFv), or an antibody mimetic such as an affibody or aptamer to any one of the cancer antigens.

In another preferred embodiment, said one or more targeting moieties of the polyplex of the invention are a DUPA moiety (HOOC(CH₂)₂—CH(COOH)—NH—CO—NH—CH(COOH)—(CH₂)₂—CO—).

In a further aspect, the invention relates to the composition or the kit-of-parts according to the invention for use in the treatment of cancer.

In a preferred embodiment, the invention relates to the composition or the kit-of-parts according to the invention for use in the treatment of cancer in a mammal. Preferably, said mammal is a human.

In a preferred embodiment, the invention relates to a method for treatment of cancer, wherein said composition or said kit-of-parts according to the invention is administered to a patient in need thereof.

In a preferred embodiment, said cancer is a solid tumor. Preferably, said cancer is a solid cancer, wherein at least one cell type included in said solid cancer expresses PSMA. Preferably said PSMA expressing cells are prostatic, neovasculature, and endothelial cells (Mhawech-Fauceglia et al., Histopathology 2007, 50:472-483; Israeli R S et al., Cancer Res 1994, 54:1807-1811; Chang S S et al, Cancer Res 1999, 59:3192-198; Morgenroth et al., Breast Cancer Research 2019, 21:116). More preferably, said PSMA expressing cells are prostatic cells.

In a preferred embodiment, said cancer is a solid PSMA expressing cancer type. PSMA expression is not restricted to prostatic cancer cells, but also expressed in other tumor types (Mhawech-Fauceglia et al., Histopathology 2007, 50:472-483; Israeli R S et al, Cancer Res 1994, 54:1807-1811; Chang S S et al, Cancer Res 1999, 59:3192-198). PSMA expression is detected preferably by immunohistochemical methods using a monoclonal antibody targeting PSMA, as e.g. described in Mhawech-Fauceglia et al., 2007. As used herein PSMA expressing cancer types have preferably a cut-off of 5% or more positive cells of the investigated tissue, wherein PSMA expression is detected by immunohistochemical methods using a monoclonal antibody targeting PSMA, preferably as described in Mhawech-Fauceglia et al.

In another embodiment, said cancer is an adenocarcinoma, more preferably a PSMA expressing adenocarcinoma.

In a preferred embodiment, said cancer is selected from the group consisting of prostate cancer, endometrial cancer, renal cell carcinoma, urothelial cell carcinoma, bladder cancer, colon carcinoma, glioblastoma (GBM), melanoma, non-small cell lung cancer, gastric cancer, oral squamous cell carcinoma, breast cancer, and metastatic cancer.

In a preferred embodiment, said cancer is a solid PSMA expressing cancer, preferably, said solid PSMA expressing cancer is selected from the group consisting of prostate cancer, urothelial cell carcinoma; bladder cancer, especially urothelial bladder carcinoma; glioblastoma (GBM); non-small cell lung cancer; breast cancer; salivary gland tumors, especially salivary adenoid cystic carcinoma; hepatocellular carcinoma; adenocarcinoma of the oesophagus, stomach, especially intestinal type of the stomach, and small intestine; and head and neck squamous cell carcinomas.

In a preferred embodiment, said cancer is a solid cancer, preferably, said solid cancer is selected from the group consisting of prostate cancer, urothelial cell carcinoma; bladder cancer, especially urothelial bladder carcinoma; glioblastoma (GBM); non-small cell lung cancer; breast cancer; salivary gland tumors, especially salivary adenoid cystic carcinoma; hepatocellular carcinoma; adenocarcinoma of the oesophagus, stomach, especially intestinal type of the stomach, and small intestine; and head and neck squamous cell carcinomas.

In another preferred embodiment, said cancer is a solid cancer selected from the group consisting of prostate cancer, urothelial cell carcinoma, bladder cancer, salivary gland tumors, especially salivary adenoid cystic carcinoma, hepatocellular carcinoma, non-small cell lung cancer, and breast cancer. More preferably, said cancer types express PSMA.

In another preferred embodiment, said cancer is a solid cancer selected from the group consisting of squamous cell carcinomas of the head and neck; adenocarcinoma of the oesophagus, stomach and small intestine, especially the intestinal type of the stomach; glioblastoma; prostate cancer; urothelial carcinoma, especially of the bladder and a combination thereof. More preferably, said cancer types express PSMA.

In a more preferred embodiment, said cancer is a solid cancer selected from the group consisting of adenocarcinoma of the stomach, especially the intestinal type of the stomach; prostate cancer; urothelial carcinoma, especially of the bladder and a combination thereof. More preferably, said cancer types express PSMA.

In an even more preferred embodiment, said cancer is a solid PSMA expressing cancer is prostate cancer or urothelial carcinoma of the bladder or a combination thereof. In an even more preferred embodiment, said cancer is prostate cancer or urothelial carcinoma of the bladder or a combination thereof.

In a most preferred embodiment, said cancer is prostate cancer. In a preferred embodiment, said polyplex included in the kit-of-parts according to the invention is administered separately from the one or more antibodies capable of modulating an immune checkpoint protein.

In one embodiment, the invention provides use of the pharmaceutical composition or kit-of-parts of the invention for the treatment of cancer, wherein said polyplex is administered to a patient in a therapeutically effective amount in combination with a therapeutically effective amount of said at least one antibody capable of modulating an immune checkpoint protein.

In a preferred embodiment, use of the kit-of-parts of the invention in the treatment of cancer comprises independent dosing of the polyplex and said at least one antibody capable of modulating an immune checkpoint protein. Said polyplex and said at least one antibody capable of modulating an immune checkpoint protein can be administered simultaneously or sequentially (consecutive), i.e. chronologically staggered. Said polyplex and said at least one antibody capable of modulating an immune checkpoint protein, as included in the kit-of-parts of the invention, can be combined prior to administration and can be administered together as composition or can be administered separately. In a more preferred embodiment, said polyplex and said at least one antibody capable of modulating an immune checkpoint protein, as included in the kit-of-parts of the invention, are administered separately.

In certain embodiments, the kits-of-parts or the composition for use according to the invention are administered by any suitable route. The kits-of-parts or the composition for use according to the invention may be administered by an intravenous, intra-brain (intracerebral), oral, intramuscular, subcutaneous, transdermal, intradermal, transmucosal, intranasal, sublingual, intraperitoneal or intraocular route. In a preferred embodiment, kits-of-parts or the composition for use according to the invention are systemically administered, i.e. enterally or parenterally. More preferably, kits-of-parts or the composition for use according to the invention are intravenously, subcutaneously or intraperitoneally administered.

In a more preferred embodiment, the kits-of-parts or the composition according to the invention are for systemic administration. More preferably, kits-of-parts or the composition according to the invention are intravenously or intraperitoneally administered, again more preferably intravenously administered.

Said polyplex and said at least one antibody capable of modulating an immune checkpoint protein of the kits-of-parts according to the invention can be administered via the same route or preferably via different routes. More preferably, said polyplex and said at least one antibody capable of modulating an immune checkpoint protein of the kits-of-parts according to the invention are administered via the same routes. In a preferred embodiment, said polyplex and said at least one antibody capable of modulating an immune checkpoint protein of the kits-of-parts according to the invention are administered sequentially or simultaneously, preferably sequentially. In a preferred embodiment, said polyplex and said at least one antibody capable of modulating an immune checkpoint protein of the kits-of-parts according to the invention are administered sequentially via different routes. More preferably, said polyplex and said at least one antibody capable of modulating an immune checkpoint protein of the kits-of-parts according to the invention are administered simultaneously via the same route.

In a preferred embodiment, said polyplex and said at least one antibody capable of modulating an immune checkpoint protein of the kits-of-parts according to the invention are administered sequentially, or simultaneously, preferably sequentially, wherein one compound (or part) of the kits of parts of the invention is administered via intraperitoneal injection and at least one other compound (or part) is administered via intravenous injection. In a preferred embodiment, said polyplex and said at least one antibody capable of modulating an immune checkpoint protein of the kits-of-parts according to the invention are administered sequentially or simultaneously, preferably sequentially, wherein the polyplex is administered via intravenous injection and the immunomodulatory antibodies are administered via intraperitoneal or intravenous injection.

In another preferred embodiment, the polyplex is administered prior to said at least one antibody capable of modulating an immune checkpoint protein. In another preferred embodiment, the polyplex and said at least one antibody capable of modulating an immune checkpoint protein are administered sequentially, wherein the polyplex is administered via intravenous injection and said at least one antibody capable of modulating an immune checkpoint protein are administered via intraperitoneal or intravenous injection, and wherein the polyplex is administered prior to the antibody.

The ratio of the amount or concentration of the polyplex to the amount or concentration the one or more antibody to be administered in the kits-of-parts or composition of the invention can be varied, e.g. in order to cope with the needs of a single patient or a patient sub-population to be treated, wherein the needs can be different due to patient's age, sex, body weight, condition etc.

The kits-of-parts of the invention further may be used as add-on therapy. As used herein, “add-on therapy” means an assemblage of said polyplex and said at least one antibody for use in therapy, wherein the subject receiving the therapy begins a first treatment regimen with one or more different parts of the kits-of-parts prior to beginning a second treatment regimen of one or more different parts of the kits-of-parts in addition to the first treatment regimen, so that not all of the reagents used in the therapy are started at the same time. For example, one or more immunomodulatory antibodies are administered to a patient already receiving the polyplex of the invention or vice versa.

The invention will now be illustrated by the following non-limiting examples.

### EXAMPLES

In the following, the invention is further illustrated by way of examples. Hereto preferred polyplexes of the present invention have been used. The synthesis of said preferred polyplex has been described in WO 2015/173824, and in particular in Examples 11 to 13, more particularly Example 12 of WO 2015/173824, which is incorporated herein by reference in its entirety. The preferred polymeric conjugate comprising PEI and PEG moiety and DUPA moiety is abbreviated herein as “PEI-PEG-DUPA” and the corresponding preferred polyplex with polyIC, is interchangeably, abbreviated as “PEI-PEG-DUPA/polyIC” or “PPD/PIC”.

### Example 1—Effects of PEI-PEG-DUPA/polyIC Alone

Secretion of the potent chemokine, IFN-7-induced protein 10 (IP-10/CXCL10), was assessed as followed: LNCaP and MCF7 cells (40,000 cells seeded per well in a 96-well plate) were treated for 5 hours with PEI-PEG-DUPA/polyIC at various concentrations (0.125, 0.25, 0.5, 1.0 μg/ml). Human IP-10 (CXCL10) secretion was quantified by ELISA assay (ABTS ELISA Development Kit, Peprotech, cat #900-K39). IP-10 secretion by LNCaP cells expressing high PSMA levels strongly increased after 5 hours of incubation with PEI-PEG-DUPA/polyIC (FIG. 1).

Cell surface expression of PD-L1 was assessed as followed: LNCaP cells (ATCC) were seeded in flat-bottom 12-well plates and treated with PEI-PEG-DUPA/polyIC at a concentration of 0.031, 0.063, or 0.125 μg/ml for 5 hours at 37° C. Then, LNCaP cells were detached using trypsin, washed and stained with anti-PD-L1-PE labelled antibody (Biolegend, cat #393607) or with an IgG1 isotype control (Biolegend, cat #400114) for 35 minutes on ice in PBS with 2% FCS, washed and analyzed by FACS instrument (FACS Aria II, BD). Treatment with 0.031, 0.063, or 0.125 μg/ml PEI-PEG-DUPA/polyIC resulted in a dose-dependent increase in PD-L1 expression (MFI=695, 725, 788, respectively) as compared to untreated control cells (MFI=579). Isotype control was used as negative control (MFI=153) (FIG. 2).

Conclusions: Secretion of the potent chemokine, IP-10, was strongly induced in the PSMA over-expressing cell line, LNCaP, following treatment with PEI-PEG-DUPA/polyIC. In MCF7 cells, which do not express PSMA, IP-10 levels were undetectable. The increase in IP-10 following PEI-PEG-DUPA/polyIC treatment provided the rationale to address the immunostimulatory properties of the inventive combination. In addition, PD-L1 cell surface expression was induced following treatment with PEI-PEG-DUPA/polyIC and was stronger than in untreated PSMA over-expressing LNCaP cells, providing the rationale for developing the combination with Nivolumab.

### Example 2—In Vitro Effects of PEI-PEG-DUPA/polyIC in Combination with Co-Stimulatory 4-1BB Antibody

The following experiments demonstrate the benefit of combination of the polyplex of the invention with an anti-4-1BB antibody over single agent treatment. IFN-γ release from PBMCs that were co-cultured with PEI-PEG-DUPA/polyIC-treated LNCaP cells was quantified.

PSMA over-expressing LNCaP cells (40,000 cells seeded per well in a 96-well plate) were treated with PEI-PEG-DUPA/polyIC at a concentration of 0.125 μg/ml for 5 hours. Anti-CD3-stimulated (clone OKT3, 5 ng/ml) or unstimulated PBMCs (200,000 cells) from healthy donors were added in the presence or absence of an antibody against 4-1BB The PBMCs were then co-cultured with LNCaP cells treated with an antibody against 4-1BB (Biolegend clone, 4B4-1; 10 μg/ml) alone or in combination with PEI-PEG-DUPA/polyIC for 16 hours (+4-1BB; FIG. 3B). As a control, PBMCs were co-cultured with LNCaP cells untreated or treated with PEI-PEG-DUPA/polyIC for 16 hours without 4-1BB ((−) 4-1BB; FIGS. 3A and B). After 16 hours of co-culture, the medium was collected and human IFN-γ was measured by ELISA assays (BD Bioscience, cat #555142).

Combination of PEI-PEG-DUPA/polyIC with anti-4-1BB antibody (+4-1BB) resulted in a significant 2-fold increase in IFN-γ secretion as compared to either treatment alone (FIGS. 3A and B). Co-culture of CD-3-stimulated PBMCs and PEI-PEG-DUPA/polyIC-treated LNCaP cells resulted in only a slight increase in IFN-γ secretion as compared to anti-CD-3-stimulated PBMCs co-cultured with untreated LNCaP (anti-CD3 alone) (FIG. 3A).

Conclusions: Co-culturing PBMCs with LNCaP cells treated with PEI-PEG-DUPA/polyIC induced the secretion of IFN-γ from PBMCs. Combining an anti-4-1BB antibody with PEI-PEG-DUPA/polyIC treatment led to a 2-fold and significant increase in IFN-γ secretion from anti-CD3-stimulated PBMCs compared to either single agent treatment.

### Example 3—In Vitro Effects of PEI-PEG-DUPA/polyIC in Combination with Checkpoint Inhibitor Anti-PD-1 Antibody

The following experiments demonstrate the benefit of combination of the polyplex of the invention with Nivolumab over single agent treatment. IFN-γ (human) release from unstimulated and anti-CD3-stimulated PBMCs that were exposed to supernatant of PEI-PEG-DUPA/polyIC-treated LNCaP cells in the presence or absence of an anti-PD-1 antibody (Nivolumab) was quantified by ELISA assays.

In the present invention, PEI-PEG-DUPA triconjugates and PEI-PEG-DUPA/polyIC polyplexes were prepared according to WO 2015/173824, Example 12; Langut et al., PNAS, Dec. 26, 2017, vol. 114, no. 52; and WO2019063705A1.

Anti-PD-1 antibody (Nivolumab) was used to test the combination of PEI-PEG-DUPA/polyIC polyplex and checkpoint blockade. Anti-PD-1 antibody (Nivolumab) was utilized either alone or in combination with PEI-PEG-DUPA/polyIC polyplex. Anti-CD3 antibody (clone OKT3, 500 ng/ml) was used to stimulate PBMCs after transfer of medium from treated LNCaP cells. Anti-PD-1 antibody (Nivolumab) was used to test the combination of PEI-PEG-DUPA/polyIC polyplex and checkpoint blockade. Human IFN-γ secretion was determined with an ELISA kit (BD Bioscience), according to the manufacturer's instructions.

LNCaP cells were seeded in a flat-bottom 96-well plate (40,000 cells per well RPMI-1640 medium). In parallel, a plate with medium alone (90 μl medium without cells) was prepared as a negative control. The next day, LNCaP cells or medium alone were treated with 0.125 g/ml of PEI-PEG-DUPA/polyIC polyplex (10 μl/well) or untreated (UT) for 5 hours at 37° C. Frozen PBMCs were thawed and allowed to recover in RPMI-1640 medium at 37° C. for at least 5 h in U-bottom 96-well plates (200,000 cells/100 μl). After 5 h, supernatant (SN) from PEI-PEG-DUPA/polyIC polyplex-treated LNCaP cells (“LNCaP Supernatant”) or PEI-PEG-DUPA/polyIC polyplex-“treated” medium (“Medium alone”) or UT untreated medium was transferred to unstimulated or anti-CD3-stimulated (OKT3, 500 ng/ml) PBMCs from healthy donors. Thereafter, PBMCs were stimulated using anti-CD3 antibody (OKT3, 500 ng/ml) or/and treated with Nivolumab (20 μg/ml) and incubated o/n at 37° C. The SN of stimulated PBMCs was collected and stored at −20° C. until analyzed by IFN-γ ELISA (BD Bioscience, cat #555142).

To model the effect of combining PEI-PEG-DUPA/polyIC polyplex with Nivolumab treatment on immune cell activation in vitro, the SN from LNCaP cells that had been treated with PEI-PEG-DUPA/polyIC polyplex was transferred to human PBMCs from a healthy donor and were treated in combination with Nivolumab, as follows: LNCaP cells were treated with PEI-PEG-DUPA/polyIC polyplex at the indicated concentrations for 5 hours. As a control, medium without cells was “treated” with PEI-PEG-DUPA/polyIC polyplex and incubated for 5 hours as well. After 5 hours, PBMCs stimulated or unstimulated with anti-CD3 antibody were treated with the following: Nivolumab alone; SN from PEI-PEG-DUPA/polyIC polyplex-treated LNCaP cells alone; PEI-PEG-DUPA/polyIC polyplex-“treated” medium alone; Nivolumab plus SN from PEI-PEG-DUPA/polyIC polyplex treated LNCaP cells; or Nivolumab plus PEI-PEG-DUPA/polyIC polyplex-“treated” medium. PBMCs were incubated o/n at 37° C. To assess PBMC activation, IFN-γ secreted from the PBMCs was quantified by ELISA.

As can be seen in FIG. 4 and Table 1, anti-CD3-stimulated PBMCs secreted low levels of IFN-γ. Addition of untreated or treated with PEI-PEG-DUPA/polyIC Medium, with or without Nivolumab, did not result in a significant increase in IFN-γ secretion. However, addition of SN from LNCaP cells led to increased IFN-γ secretion (1788.6 μg/ml) which further elevated with SN from PEI-PEG-DUPA/polyIC-treated LNCaP cells (“LNCaP Supernatant”) (2893.4 μg/ml). Secretion of IFN-γ from anti-CD3-stimulated PBMCs with supernatant from UT cells in the presence or absence of Nivolumab was not significantly different (2042.3 μg/ml). However, IFN-γ secretion by was strongly enhanced (2-fold) upon combination of Nivolumab and supernatant from PEI-PEG-DUPA/polyIC-treated LNCaP cells (4448.2 μg/ml).

As can be seen in FIG. 4 and Table 1, CD3-stimulated PBMCs that received SN from PEI-PEG-DUPA/polyIC polyplex-treated LNCaP cells (“LNCaP Supernatant”) induced the secretion of higher concentrations of IFN-γ (2893.4 μg/ml) than PBMCs that received SN from untreated (UT) cells (1788.6 μg/ml). IFN-γ secretion was further significantly enhanced by the addition of Nivolumab to PBMCs that received medium from PEI-PEG-DUPA/polyIC polyplex-treated LNCaP (4448.2 μg/ml). PBMCs that received SN from untreated cells plus Nivolumab secreted lower levels of IFN-γ (2042.3 μg/ml). PEI-PEG-DUPA/polyIC polyplex-“treated” medium (without cells) alone (“Medium alone”) or in combination with Nivolumab did not result in an increase in IFN-γ secretion as compared to UT medium.

Conclusions: Combination of PEI-PEG-DUPA/polyIC polyplex with Nivolumab resulted in significantly increased activation of PBMCs, as seen by increased IFN-γ secretion from anti-CD3-stimulated PBMCs treated with the combination, as compared to PBMCs treated either with Nivolumab or with supernatant from PEI-PEG-DUPA/polyIC-treated LNCaP cells alone. PBMCs are activated by the cytokines that are secreted from the PEI-PEG-DUPA/polyIC-treated LNCaP cancer cells, rather than by PEI-PEG-DUPA/polyIC polyplex treatment alone.

TABLE 1
Combination of PEI-PEG-DUPA/polyIC polyplex and Nivolumab
increases PBMC activation as demonstrated by IFN-γ ELISA
Medium + PEI-PEG-DUPA/polyIC polyplex (Medium alone)
IFN-γ values (pg/ml) from PBMCs treated with Medium and/or
CD3/Nivolumab + PEI-PEG-DUPA/polyIC polyplex

| Treatment | | | | |
|---|---|---|---|---|
| of Medium | Medium | Nivolumab | CD3 | CD3 + Nivolumab |
| UT | 0 | 33.9 | 499.8 | 714.2 |
| 0.125 μg/ml PEI- | 66.2 | 38.7 | 499.8 | 963.4 |
| PEG-DUPA/polyIC | | | | |

LNCaP SN + PEI-PEG-DUPA/polyIC polyplex (“LNCaP Supernatant”)
IFN-γ values (pg/ml) from PBMCs treated with LNCaP SN and/or
CD3/Nivolumab + PEI-PEG-DUPA/polyIC polyplex

| Treatment | Medium | | | +CD3 + |
|---|---|---|---|---|
| of LNCaP | (Supernatant) | Nivolumab | +CD3 | Nivolumab |
| UT | 0 | 0 | 1788.6 | 2042.3 |
| 0.125 μg/ml PEI- | 0 | 206.5 | 2893.4 | 4448.2 |
| PEG-DUPA/polyIC | | | | |

1. A kit-of-parts or composition comprising
a. a polyplex comprising a double stranded RNA (dsRNA) and a polymeric conjugate, wherein said polymeric conjugate comprises a polyethyleneimine (PEI), one or more polyethylene glycol (PEG) moieties and one or more targeting moieties, wherein said PEI is covalently bound to one or more PEG moieties, and each of said one or more PEG moieties is linked to one of said one or more targeting moieties, and
wherein each of said one or more targeting moieties is capable of binding to prostate specific membrane antigen (PSMA); and
b. at least one immune checkpoint modulator, wherein said at least one immune checkpoint modulator is capable of modulating an immune checkpoint protein.

2. The composition or the kit-of-parts according to claim 1, wherein said immune checkpoint modulator is an agonistic or antagonistic antibody.

3. The composition or the kit-of-parts according to claim 1 or 2, wherein said immune checkpoint protein is selected from the group consisting of 4-1BB, PD-1, PD-L1, and PD-L2.

4. The composition or the kit-of-parts according to any one of the preceding claims, wherein said immune checkpoint protein is 4-1BB or PD-1.

5. The composition or the kit-of-parts according to any one of the preceding claims, wherein said at least one immune checkpoint modulator is an antibody selected from the group consisting of an anti-4-1BB antibody, anti-PD-1 antibody, PD-L1 antibody, and anti-PD-L2 antibody, preferably an anti-4-1BB antibody or anti-PD-1 antibody.

6. The composition or the kit-of-parts according to any one of the preceding claims, wherein said at least one immune checkpoint modulator is a mixture comprising (i) a modulator capable of modulating the immune checkpoint protein 4-1BB and (ii) a modulator capable of modulating an immune checkpoint protein selected from the group consisting of PD-1, PD-L1, and PD-L2, preferably PD-1.

7. The composition or the kit-of-parts according to any one of the preceding claims, wherein said one or more targeting moieties is a urea based PSMA peptidase inhibitor.

8. The composition or the kit-of-parts according to claim 7, wherein said one or more targeting moieties is a glutamate-urea based PSMA peptidase inhibitor.

9. The composition or the kit-of-parts according to claim 8, wherein said glutamate-urea based PSMA peptidase inhibitor has a molecular weight of less than about 2000 g/mol, preferably less than about 1000 g/mol, more preferably less than about 500 g/mol.

10. The composition or the kit-of-parts according to any one of the preceding claims, wherein said one or more targeting moieties comprises or preferably consists of a DUPA derivative (HOOC(CH₂)₂—CH(COOH)—NH—CO—NH—CH(COOH)—CH₂—) or DUPA moiety (HOOC(CH₂)₂—CH(COOH)—NH—CO—NH—CH(COOH)—(CH₂)₂—CO—).

11. The composition or the kit-of-parts according to any one of the preceding claims, wherein said PEI is covalently bound to one, two or three PEG moieties, preferably to one or three PEG moieties.

12. The composition or the kit-of-parts according to any one of the preceding claims, wherein said polyethyleneimine (PEI) is linear polyethyleneimine (LPEI).

13. The composition or the kit-of-parts according to any one of the preceding claims, wherein said dsRNA is polyinosinic-polycytidylic acid double stranded RNA (polyIC).

14. The composition or the kit-of-parts for use in the treatment of cancer.

15. The composition or the kit-of-parts for use according to claim 14, wherein said cancer is a solid cancer, wherein preferably said solid cancer is selected from the group consisting of prostate cancer, urothelial cell carcinoma; bladder cancer; glioblastoma (GBM); non-small cell lung cancer; breast cancer; salivary gland tumors; hepatocellular carcinoma; adenocarcinoma of the oesophagus, stomach and small intestine; and head and neck squamous cell carcinomas.
